(19) United States
(12) Patent Application Publication
(10) Pub. No.: US 2025/0084379 A1
(43) Pub. Date: Mar. 13, 2025

(54) Cell Cultivation Methodology

(71) Applicant: Ronawk, Inc., Olathe, KS (US)

(72) Inventors: Adam J. Mellott, Parkville, MO (US); Jacob G. Hodge, Olathe, KS (US); Heather E. Decker, Kansas City, KS (US)

(21) Appl. No.: 18/726,671

(22) PCT Filed: Jan. 4, 2023
(86) PCT No.: PCT/US2023/010087

§ 371 Date: Jul. 3, 2024

Related U.S. Application Data
(60) Provisional application No. 63/296,267, filed on Jan. 4, 2022.

(51) Int. Cl. C12N 5/0775; C12N 5/071

(52) U.S. Cl. CPC C12N 5/0667; C12N 5/0629; C12N 2513/00; C12N 2539/00

(57) ABSTRACT

Method of cultivating cells utilizing one or more interlocking porous hydrogel block (IPHB) as a scaffolding for cell growth and propagation are provided. The IPHB(s) include a continuous polymeric matrix material and a network of microporous channels and/or chambers extending throughout the continuous polymeric matrix material. The methods include seeding a first IPHB with one or more cells of interest, feeding the one or more cells of interest with a first culture media, and allowing the one or more cells of interest to propagate throughout the network of microporous channels and/or chambers, expanding the initial scaffolding by interlocking a second IPHB to the first IPHB, and allowing the one or more cells of interest to propagate from the first IPHB into the second IPHB, and feeding the one or more cells of interest located inside the second IPHB with the first culture media or a second culture media.

### CROSS-REFERENCE TO RELATED APPLICATIONS

This application claims priority to U.S. Provisional Application No. 63/296,267, filed Jan. 4, 2022, which is expressly incorporated by reference herein in its entirety.

### TECHNICAL FIELD

Embodiments of the presently-disclosed invention relate generally to cell cultivation methods utilizing one or more interlocking porous hydrogel blocks (IPHB) that can be interlocked with each other to provide continuous 3D growth of a variety of cells and/or tissues. The IPHBs provide increased flexibility to practice a wide variety of unique cell cultivation methods (e.g., chain cultivation of cells, simultaneous multi-cell cultivation, etc.).

### BACKGROUND

Cells (e.g., human, animal, plant, bacteria, fungi) are cultivated traditionally on plastic substrates, such a petri dishes, mono-plates, well-plates, flasks, and scaffolds. However, each of these cell culture vessels are discrete and finite or limited in surface area for cell propagation. For example, these in vitro expansion systems include the use of rigid, 2D plastic culture vessels to expand the cells as a monolayer and require multiple protease-dependent sub-culturing (passaging) events to achieve large enough cell quantities. Evidence suggests that traditional 2D culture systems are not ideal for stem cell expansion and can result in a loss of multipotency, reduce viability, induce senescence and decrease secretion of regenerative factors. These types of vessels, moreover, do not mimic the internal environments from which the cells of interest naturally grow (e.g., human body from which different cell types arise). While these cell culture vessels enable easy cultivation of cells, the cells must be dissociated using a toxin to the cells and transferred to additional cell culture vessels upon outgrowing the original cell culture vessel. This process, as referenced above, is known as sub-culturing and is also known as passaging. The process of propagating cells is time-consuming, prone to error, laborious, expensive. As primary cells derived directly from tissue sub-cultured over and over, these primary cells eventually lose native characteristics (phenotype) and function. At a certain point, primary cells will stop dividing and will no longer function as they did when first extracted from primary tissue. This ceasing of function is known as senescence, and may limit the lifetime of propagation of cells from an initial sample of a primary cell of interest.

Therefore, there remains a need in the art for cell cultivation methods that provide one or more of the following: mimics internal environment, eliminates the need for sub-culturing of cells, enables continuous cell growth while optionally simultaneously harvesting grown cells, and enables simultaneous multi-cell cultivation where interfaces between different cells of interest may be produced for further use and/or analysis.

### SUMMARY OF INVENTION

One or more embodiments of the invention may address one or more of the aforementioned problems. Certain embodiments according to the invention provide a method of cultivating cells. The method may comprise the following steps: (i) providing an initial scaffolding comprising a first interlocking porous hydrogel block (IPHB), wherein the first IPHB comprises a three-dimensional (3D) macrostructure defined by a continuous polymeric matrix material and a network of microporous channels and/or chambers extending throughout the continuous polymeric matrix material, and wherein the 3D macrostructure comprises a top surface, a bottom surface, and a thickness defined by at least one side edge extending from the top surface to the bottom surface, and wherein the 3D macrostructure structure includes at least one interlocking-male component and at least one interlocking-female component; (ii) seeding the first IPHB with one or more cells of interest; (iii) feeding the one or more cells of interest with a first culture media, and allowing the one or more cells of interest to propagate throughout the network of microporous channels and/or chambers; (iv) expanding the initial scaffolding by interlocking a second IPHB to the first IPHB, wherein the at least one interlocking-male component or at least one interlocking-female component of the first IPHB is joined to a corresponding interlocking-male component or corresponding interlocking-female component of the second IPHB; and (v) allowing the one or more cells of interest to propagate from the first IPHB into the second IPHB, and feeding the one or more cells of interest located inside the second IPHB with the first culture media or a second culture media.

### BRIEF DESCRIPTION OF THE DRAWING(S)

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout, and wherein:

FIG. 1 is a visual illustration of a method in accordance with certain embodiments of the invention;

FIG. 2 illustrates two (2) separate IPHBs in accordance with certain embodiments of the invention;

FIG. 3 illustrates three (3) interlocked IPHBs in accordance with certain embodiments of the invention;

FIG. 4 depicts top and side views of an IPHB and illustrates the continuous polymeric matrix material and the network of microporous channels and/or chambers extending throughout the continuous polymeric matrix material, which are exposed at the surfaces of the IPHB, in accordance with certain embodiments of the invention;

FIG. 5 illustrates a IPHB including an interlocking-male component located on a top surface and an interlocking-female component protruding into the bottom surface in accordance with certain embodiments of the invention;

FIG. 6 illustrates another IPHB including an interlocking-male component located on a top surface and an interlocking-female component protruding into the bottom surface in accordance with certain embodiments of the invention;

FIG. 7 illustrates cell migration from a first IPHB to a second IPHB when interlocked together in accordance with certain embodiments of the invention;

FIGS. 8A-8F illustrate cell growth patterns within a microchannel in accordance with certain embodiments of the invention;

FIG. 9 is a schematic illustrating the nomenclature and conceptual cell migration of a plurality of IPHBs in accordance with certain embodiments of the invention;

FIG. 10 is a schematic illustrating additional options for adding a varying number and positioning of additional IPHBs to those of FIG. 9;

FIG. 11 is a schematic for a infiltration assay configuration of a series of interlocked empty IPHBs with four (4) seeded IPHBs interlocked as side-chains to the series of interlocked empty IPHBs;

FIG. 12 is a schematic for a dual-infiltration assay configuration of a series of interlocked empty IPHBs with a first set of four (4) IPHBs seeded with a first cell type interlocked as side-chains to the series of interlocked empty IPHBs and a second set of four (4) IPHBs seeded with a second cell type, which may produce or secrete a therapeutic agent interlocked as side-chains on an opposite side of the series of interlocked empty IPHBs;

FIG. 13 illustrates a multi-infiltration assay configuration in which eight IPHBs are attached as side-chains to a series of interlocked empty IPHBs, in which each IFPB attached as a side-chain is seeded with a different cell type, each of which may produce or secret a different therapeutic agent;

FIG. 14 illustrates a migration assay configuration in which a series of seeded IPHBs have eight (8) side-chains of unseeded or empty IPHBs attached thereto;

FIG. 15 illustrates a dual-migration assay configuration in which a series of seeded IPHBs include a first group of IPHBs are seeded with a first cell type and a second group of IPHBs are seeded with a second cell type, in which eight (8) side-chains of unseeded or empty IPHBs are attached thereto;

FIG. 16 illustrates a multi-migration assay configuration in which a series of seeded IPHBs, in which each IPHB is seeded with a different cell type, and eight (8) side-chains of unseeded or empty IPHBs are attached thereto;

FIG. 17 illustrates an 8-chamber omni tray filled with a plurality of IPHBs and spacers, in which each chamber is filled with a group of four (4) interlocked IPHBs seeded with a different cell type (e.g., each chamber has its own cell type therein);

FIG. 18 illustrates an 8-chamber omni tray filled with a plurality of IPHBs and spacers, in which the IPHBs are configured as a one-way paracrine signaling assay;

FIG. 19 illustrates an 8-chamber omni tray filled with a plurality of IPHBs and spacers, in which the IPHBs are configured as a two-way paracrine signaling assay;

FIG. 20 illustrates an 8-chamber omni tray filled with a plurality of IPHBs and spacers, in which the IPHBs are configured as paracrine signaling assay;

FIG. 21 illustrates an 8-chamber omni tray filled with a plurality of IPHBs and spacers, in which the IPHBs are configured as a four-way paracrine signaling assay;

FIG. 22A illustrates the macrostructure of the hydrogel system, in which (i) the left image is a single photographic image of the ˜1-cm³ 3D hydrogel system; (ii) the middle image is a photographic image of four hydrogels connected to each other within a six-well plate; and (iii) the right image is a photographic image of four hydrogels, connected with annotations depicting the migration of cells out/from an initially seeded hydrogel into/toward supplementally attached hydrogels without cells;

FIG. 22B illustrates the microstructure of the hydrogel system, in which (i) the left image is a confocal microscopy image of a cross-section of the internal structure of the hydrogel depicting adipose-derived mesenchymal stem/stromal cell (ASCs) migrating and proliferating within the porous architecture where (a) the small white arrow indicates polymeric struts of the hydrogel, (b) Blue=Hoechst, (c) green=phalloidin, and (d) red=Mitotracker; (ii) the middle image is a fluorescent image of ASCs lining an individual pore within the microstructure of the hydrogel system where (a) Blue=Hoechst, (b) green=wheat germ agglutinin, and (c) Red=Mitotracker; and (iii) the right image is a confocal microscopy z-stack image of ASC migrating from an initially seeded hydrogel (Hydrogel #1) into a newly attached hydrogel (Hydrogel #2), in which the large white arrows indicate directionality of ASC migration, while the white dashed line indicates the junction of the two attached hydrogels;

FIG. 22C illustrates three images depicting a 3D z-stack of images within a single pore channel to highlight (middle image) the 3D networks formed by ASCs and (right image) cellular extension protruding from the cells, while the left image is a low-magnification image of the entire stained hydrogel where all images were acquired by Nikon on their AXR Confocal Imaging System in which Blue=Hoechst, Green=phalloidin, and Pink=Mitotracker;

FIGS. 23A-23B illustrate results of ASCs that were seeded at passage 1 (P1) within the 3D hydrogel system or traditional 2D culture, in which ASCs were continuously subcultured and assessed at P2, P6 and P10 for 2D culture while the ASCs in the 3D hydrogel system were compared with their respective 2D counterparts via passage-equivalent time points. FIG. 23A illustrates that at each respective time point (P2, P6 and P10), representative images of 2D (leftmost columns) and 3D (right-most columns) cultured ASCs stained for either CD73 (left), CD90 (middle) or CD105 (right) are depicted. CD marker staining is denoted by green in the images. Samples were counterstained with Hoechst (blue) and phalloidin (not shown). FIG. 23B illustrates quantification of imaging data performed and total percentage of positive cells denoted by box-and-whiskers plots for each marker, in which each individual point indicates quantification of a single image of ASCs in 2D (black circles) or 3D (teal diamonds) cultures using a 20× objective. Samples were analyzed in quadruplicate (n=4). Scale bar=100 μm. Error bars are standard error of the mean, while *p<0.05; **p<0.01; ***p<0.001; ****p<0.0001;

FIGS. 24A-24N illustrate the reduced induction of senescence in 3D hydrogel over time as compared to a traditional 2D culture system, in which ASCs were seeded at passage 1(P1) within the 3D hydrogel system or traditional 2D culture and the ASCs were continuously subcultured and assessed at P2, P6 and P10 for 2D culture. The ASCs in the 3D hydrogel system were compared with their respective 2D counterparts via passage-equivalent time points. Three additional hydrogels were added to each individual hydrogel at the 2-week mark and left for the remainder of the culture period to provide adequate surface area for continuous cell growth. FIG. 24A illustrates that at each respective time point (P2, P6 and P10), representative images of 2D (top row) and 3D (bottom row) cultured ASCs stained for β-galactosidase (green) are depicted. Samples were counterstained with Hoechst (blue) and phalloidin (not shown). FIG. 24B illustrates the quantification of imaging data performed and total percentage of positive cells denoted by box-and-whiskers plots for each marker. Each individual point indicates quantification of a single image of ASCs in 2D (black circles) or 3D (teal diamonds) using a 20× objective. Samples were analyzed in quadruplicate (n=4). Scale bar=100 μm. Error bars are standard error of the mean and ***p<0.001; ****p<0.0001; and

FIGS. 25A-25D illustrate retention of wound healing capacity in adipose-derived mesenchymal stem/stromal cell-conditioned media (ASC-CM) from 2D and 3D from each respective time point that was used to treat keratinocytes, which were then assessed for changes in their migratory, metabolic and proliferative activity. FIGS. 25A and 25B illustrate migratory activity that was assessed via a scratch assay, in which FIG. 25A illustrates whole-well image scans that were acquired, and representative images of the wound images are provided, wherein white solid lines denote original wound boundaries and black solid lines outline the remaining wound area. FIG. 25B illustrates the average wound area after 22 h was determined and performed in triplicate. FIG. 25C illustrates metabolic activity that was quantified via PrestoBlue and is displayed as an average value of relative fluorescent units. FIG. 25D illustrates proliferative activity that was quantified via PicoGreen and the average cell number was determined. Keratinocytes treated with 2D ASC-CM are denoted with black bars; keratinocytes treated with 3D ASC-CM are denoted with teal bars. Scale bar=500 μm. *p<0.05; **p<0.01; ***p<0.001; ****p<0.0001. Error bars are standard error of the mean.

### DETAILED DESCRIPTION

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The presently-disclosed invention relates generally to cell cultivation methods utilizing a platform where cells of interest may continuously propagate on a substrate (e.g., a 3D scaffold) without the need for sub-culture. In accordance with certain embodiments of the invention, the methods of cultivating cells of interest utilize one or more interlocking porous hydrogel blocks (IPHB) that can be interlocked with each other to provide continuous 3D growth of a variety of cells and/or tissues. In this regard, the IPHBs provide a substrate that is modular, and connect or link together, for example, in a manner similar to the attachment of puzzle pieces. Cells grown on one IPHB may migrate, and propagate on a second IPHB when the second IPHB is linked or connected to the original IPHB. Additional IPHBs (e.g., scaffolding substrates) may be connected or linked to the original IPHB or IPHBs linked or connected to the original IPHB. Beneficially, therefore, cells may continue propagating as long as additional IPHBs are linked or connected together. As such, the system of interconnected IPHBs may define an aggregate 3D network of microporous channels and/or chambers extending in a continuous manner throughout the entirety of the interconnected IPHBs (e.g., each IPHB has its own 3D network of microporous channels and/or chambers, which may be the same or different from any of the others).

In accordance with certain embodiments, the methods of cell cultivation are devoid of any sub-culturing step, such methods of avoiding sub-culture preserves cell characteristics for twice as long or greater than when cells are grown in a vessel that requires sub-culturing. The modular IPHBs (e.g., substrates) may be two-dimensional (2D) or 3D, while a 3D configuration may be more desirable. As noted above and discussed in more detail below, the IPHBs may contain void spaces, channels, or be permeable to gas or liquid substrates. In accordance with certain embodiments of the invention, the continuous polymeric material of the IPHBs may be made mostly or exclusively hydrogel materials comprising or consisting of synthetic polymers, biologically derived polymers, and/or tissue derived components such extracellular matrix. By use of hydrogel materials, the IPHBs may be individually tailored to have characteristics that mimic the environment of virtually any environment within, for example, the human body to enable cells to grow more natively, and maintain cell characteristics for a longer period than when grown in cell culture vessel that requires sub-culturing. Moreover, different IPHBs may be combined together (e.g., interlocked) to allow different types of cells to mix to form tissues or produce multi-cell organoids. IPHBs may be fixed in place to preserve cells for analysis or substrates may be degraded to release cells for analysis in accordance with certain embodiments of the invention.

In accordance with certain embodiments of the invention, the methods of cultivating cells may enable end-users to continuously culture virtually any cell type without stopping and without sub-culturing. This, beneficially, enables continuity of experiments and continuity of cell production not realized by traditional platforms. Moreover, the methods of cultivating cells minimizes human interaction, which reduces human errors, contamination risks, and other inconsistencies since the need for sub-culturing is eliminated. Furthermore, the methods utilizing the IPHBs, in accordance with certain embodiments of the invention, may be compatible with current cell culture trays and plates, which means these methods may be used in automated systems that can transfer standard cell culture trays and plates between incubators and liquid handlers for dispensing and aspirating cell media. With less manual operation and sub-culturing, the methods disclosed herein may also dramatically reduce the consumption of consumables including media and plasticware. The methods utilizing the IPHBs, in accordance with certain embodiments of the invention, also permits uniform nutrient and gas exchange through the IPHB (e.g., hydrogel substrate) based on the microchannel architecture built into the IPHB. These methods utilizing IPHBs, for example, enable customization to facilitate growth of specific cell types, such as the hydrogel material forming the continuous polymeric network and/or the structure of the network of microporous channels and/or chambers extending throughout the continuous polymeric matrix material of the IPHB. Moreover, methods in accordance with certain embodiments of the invention permit the culture of multiple different cell types in combination, permits cells to secret and form extracellular matrices that can be harvested as raw materials for therapeutic or diagnostic purposes, promotes the secretion of biological products from cells into the luminal space of the microchannels for collection.

In accordance with certain embodiments of the invention, the IPHBs permit perfusion of liquid media and/or gases through the microchannels thereof, and permits cells to organize according to the design of the microarchitecture for the purpose of forming specific tissue structures. Beneficially, methods of cell cultivation in accordance with certain embodiments of the invention overcomes the problem of spheroid necrosis by enabling spheroids to unwind within the microchannels and form cylinders and other higher-order structures. In this regard, this present methodology facilitates the formation of native microenvironments that promote cell proliferation, migration, viability, and function. Moreover, the flexible and robust nature of the methods utilizing the IPHB-based system may permit custom growth of cell cultures.

In accordance with certain embodiments of the invention, the methods of cell cultivation may be applied to the production of biologics, such as exosomes, extracellular vesicles, growth factors, monoclonal antibodies, peptides, proteins, viral particles, oligonucleotides, and organelles. In this regard, cells that produce or secrete a biologic of interest may be cultivated as disclosed herein, and the produced or secreted biologic may be isolated for further processing, use, and/or analysis. Additional applications for which methods of cell cultivation may be applied include, for example, tissue formation, organoid formation, filtration, regenerative medicine, auto-graft generation, meat production, cell culture, plant culture, protein production, physiological modeling, infectious disease, wound healing, cellular reprogramming, disease modeling, cancer research, bioreactor, mass cell production, and microenvironment formation.

As noted above, the presently-disclosed invention relates generally to methods of cultivating cells. The method, in accordance with certain embodiments of the invention may comprise the following steps: (i) providing an initial scaffolding comprising a first interlocking porous hydrogel block (IPHB), wherein the first IPHB comprises a three-dimensional (3D) macrostructure defined by a continuous polymeric matrix material and a network of microporous channels and/or chambers extending throughout the continuous polymeric matrix material, and wherein the 3D macrostructure comprises a top surface, a bottom surface, and a thickness defined by at least one side edge extending from the top surface to the bottom surface, and wherein the 3D macrostructure structure includes at least one interlocking-male component and at least one interlocking-female component; (ii) seeding the first IPHB with one or more cells of interest; (iii) feeding the one or more cells of interest with a first culture media, and allowing the one or more cells of interest to propagate throughout the network of microporous channels and/or chambers; (iv) expanding the initial scaffolding by interlocking a second IPHB to the first IPHB, wherein the at least one interlocking-male component or at least one interlocking-female component of the first IPHB is joined to a corresponding interlocking-male component or corresponding interlocking-female component of the second IPHB; and (v) allowing the one or more cells of interest to propagate from the first IPHB into the second IPHB, and feeding the one or more cells of interest located inside the second IPHB with the first culture media or a second culture media.

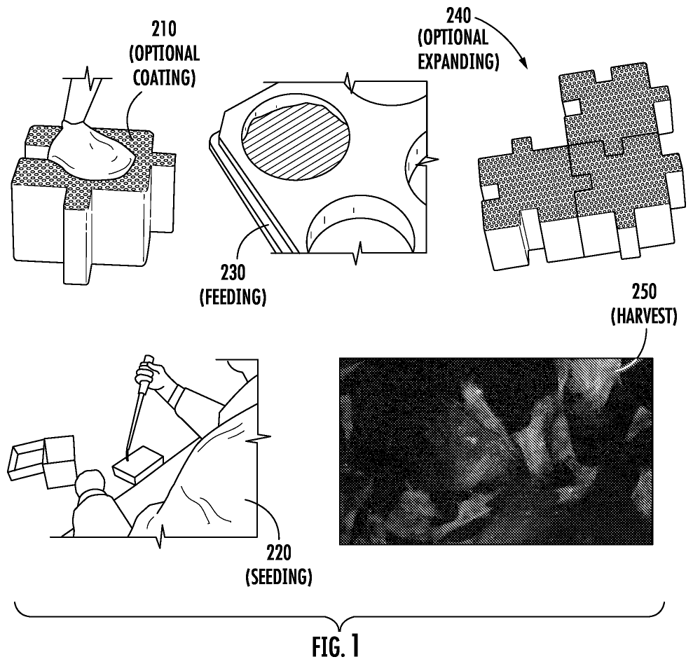

FIG. 1, for instance, illustrates one example method of cultivating cells 200 including an optional step 210 of coating a first IPHB 1 (e.g., a primary or seeding block) with a compatibilizer that may promote or facilitate adhesion of the cells of interest to the polymeric matrix material within the inside of the first IPHB (e.g., the walls defining the network of microporous channels and/or chambers extending throughout the continuous polymeric matrix material). As shown in FIG. 1, the coating step with the compatibilizer may be performed simply by applying a flowable or liquid composition including the compatibilizer to the top surface of the first IPHB and allowing the composition to diffuse and/or flow into and through the network of microporous channels and/or chambers extending throughout the continuous polymeric matrix material. In accordance with certain embodiments of the invention, a gas or combination of the liquid composition and gas may be conveyed through the first IPHB for the purposes of changing the texture of the network of microporous channels and/or chambers for facilitating cell adhesion, cell migration, cell proliferation, or other cell behavior and function. In this regard, the first IPHB may be bathed or soaked in the composition having the compatibilizer to ensure desirable coating of the network of microporous channels and/or chambers with the compatibilizer. FIG. 1 also illustrates that the operation 220 of seeding the first IPHB with one or more cells of interest (e.g., a first type of cells for culturing). The nature of the cells is generally not critical as the IPHB is agnostic and will accept any cell line. Step 230 may comprise feeding the now seeded cells of interest that are present within the first IPHB. In this regard, one or more culture mediums may be used according with the desired protocol for growth of the seeded cells. Again, the feeding operation may be conducted by applying the culture media to the top surface of the IPHB and allowing the culture media to diffuse and/or flow through the IPHB. Additionally or alternatively, the IPHB may be soaked or submerged in the culture media. As shown in FIG. 1, optional step 240 may comprise expanding the initial scaffolding associated with the first IPHB by interlocking a second IPHB and a third IPHB to the first IPHB. Step 250 comprises harvesting the cultured cells from within the IPHB(s). Depending on the nature of the hydrogel material forming the continuous polymeric matrix material, harvesting may comprise degrading the hydrogel material to expose the cells, or the cells may be flushed out with a retrieval reagent in liquid form that is passed through the IPHB(s).

In accordance with certain embodiments of the invention, the method may further comprising a step of harvesting at least a portion of the one or more cells located throughout the network of microporous channels and/or chambers of the first IPHB. The step of harvesting at least a portion of the one or more cells located throughout the network of microporous channels and/or chambers of the first IPHB may comprise flushing them out of the first IPHB with a fluid medium. Depending on the IPHB being used, end-users may utilize, for example, Trypsin and Accutase based products to remove cells from the network of microporous channels and/or chambers. In accordance with certain embodiments of the invention, an enzymatic reagent physically cuts the bonds in the hydrogel material forming the continuous polymeric matrix material to degrade the IPHB and gently release the cells located within the network of microporous channels and/or chambers. Additionally or alternatively, the step of harvesting at least a portion of the one or more cells located throughout the network of microporous channels and/or chambers of the first IPHB may comprise degrading the 3D macrostructure of the first IPHB. As noted herein, degradable materials may include naturally occurring biopolymers, such as collagen, hyaluronan, gelatin, and nucleic acids. These type of materials, or combinations of these materials can be degraded with corresponding enzymes such as collagenase (e.g., works for collagen and gelatins), hyaluronase, and Dnase/Rnase.

In accordance with certain embodiments of the invention, the methods may optionally comprise a step of coating an interface between the continuous polymeric matrix material and the network of microporous channels and/or chambers extending throughout the continuous polymeric matrix material with a compatibilizer, such as described and disclosed herein. The compatibilizer, for example, may be selected to promote adhesion of a primary cell of interest to the IPHB. In this regard, the coating step would occur prior to seeding of the IPHB.

In accordance with certain embodiments of the invention, the IPHBs can be interlocked with each other to provide continuous 3D growth of a variety of cells and/or tissues. Hydrogels are insoluble polymer matrices that can be engineered to hold up to 96% water content by mass, such as up to 40, 50, 60, 70, 80, 90, and 95% water content by mass). A variety of different polymers can be used individually or in combination to create unique hydrogels. Through cross-linking of polymers via light, temperature shift, or chemical reaction, hydrogels can be tailored to exhibit different mechanical properties, diffusion gradients, osmotic pressures, chemical formulations, and structures such as pores and fibers of varying shapes and sizes. Hydrogels may also be degradable or non-degradable. Hydrogels are versatile in their ability to be used in different applications, such as soft contact lenses to provide optics to correct a patient's vision. Hydrogels have been used in wound healing applications as a dressing and have also been used as bioinks in Life Science applications to create unique structural scaffolds for micro-fluidic experiments or provide a substrate for cells to be cultured on or in.

The IPHBs, in accordance with certain embodiment of the invention, may be joined together or interlocked together via at least one interlocking-male component and at least one interlocking-female component. For example, the at least one interlocking-male component of a first IPHB is configured to be received within a corresponding at least one interlocking-female component of a second IPHB. In addition to the joining of the IPHBs via their 3D macrostructure, the microstructure of network of microporous channels and/or chambers (e.g., void spaces). Most hydrogels are solid materials. However, by introducing void spaces and microchannels into the hydrogel, liquid, gas, and cell migration can be directed for the purpose of expanding the hydrogels together to form a continuous substrate. This feature, in accordance with certain embodiments of the invention, may be particularly beneficial since microstructure of network of microporous channels and/or chambers (e.g., void spaces) allow for a second medium to be used to interlock the IPHBs (e.g., hydrogels) together, and create a larger or expanded material for cell and/or tissue growth.

Hydrogels may be formed by crosslinking any synthetic polymer, biological polymer, tissue component (derived from human, animal, plant, or combination thereof), or combination thereof in the presence of water using a free-radical mediated reaction (e.g., photo reaction, chemical reaction) or reaction as a result of change in temperature.

The IPHBs (e.g., hydrogels), in accordance with certain embodiments of the invention, may be suitable for a variety of applications, such as producing or growing cell cultures, biologics, exosomes, extracellular vesicles, growth factors, monoclonal antibodies, peptides, proteins, viral particles, oligonucleotides, organelles, organoid formation, plant growth, drug delivery, tissue formation, ex vivo modeling, electrical conduction, wound healing, cellular reprogramming, filtration, optics, microfluidics, custom network of microchannels, custom scaffold architecture construction, custom extracellular matrix derived scaffolds, dissolvable hydrogels, custom tissue formation, accepts patient cells, custom configurations, modular, and microenvironment manipulation.

In accordance with certain embodiments of the invention, the IPHBs allow for cells to grow in a more native physiological-like environment compared to culture in a 2D plastic cell culture vessel. For instance, the IPHBs may be tuned or configured to mimic an original tissue environment from which specific cells arise and grow, unlike plastic cell culture vessels and other technologies that are not customizable and modular. Additionally, certain embodiments of the invention provide for the combination of multiple IPHB (e.g., hydrogel) substrates to be joined that are like or unlike to form an expanded continuous hydrogel substrate for cell production and/or biologics production. For example, like or unlike hydrogels (e.g., IPHB) may be joined together to create unique and custom microenvironments for cells to grow in, unlike other cell culture vessels or technologies. Moreover, the IPHBs can allow for the formation of spheroids and organoids without developing a necrotic core inside the IPHB's network of microchannels. For example, the IPHBs, in accordance with certain embodiments of the invention, allows spheroids and organoids to unwind and form tubes, cylinders, and other sophisticated structures where nutrients and gases may evenly diffuse to cells within the IPHB (e.g., hydrogel). Beneficially, for instance, the IPHBs may permit even nutrient and gas exchange for healthy cell growth unlike other technologies that claim to mass produce cells. In accordance with certain embodiments of the invention, the IPHBs may be modified to suite a wide variety of different cell types. In accordance with certain embodiments of the invention, the IPHBs enables cells to secrete extracellular matrix and create natural microenvironments that promote cell growth, migration, viability, and function. Still further, the IPHBs beneficially eliminate the need to sub-culture cells. Moreover, the use of the IPHBs is easy to use as they can be provided in a pre-formed format, and does not require sophisticated changes in temperature, pH, or chemical exposure to use. The IPHBs, for example, may enable users to achieve one or more of the following: grow custom cell cultures, grow multiple cell types in parallel or sequence, combine cell cultures to create complex tissues, mass produce cells without ever stopping production of cells, use the same substrates to produce cells from the benchtop all the way through clinical trials and for industrial production, and use less media and fewer consumables than present technologies, reduce human error and risks of contamination by reducing or eliminating human touch points in the production of cells. In accordance with certain embodiments of the invention, the IPHBs provide a modular platform for any of the above-referenced applications.

Certain embodiments according to the invention provide an interlocking porous hydrogel block (IPHB) comprising a three-dimensional (3D) macrostructure defined by a continuous polymeric matrix material and a network of microporous channels and/or chambers extending throughout the continuous polymeric matrix material. The 3D macrostructure may comprise a top surface, a bottom surface, and a thickness defined by at least one side edge extending from the top surface to the bottom surface, in which the 3D macrostructure includes at least one interlocking-male component and at least one interlocking-female component. In accordance with certain embodiments of the invention, the at least one interlocking-male component of a first IPHB is configured to be received within a corresponding at least one interlocking-female component of a second IPHB.

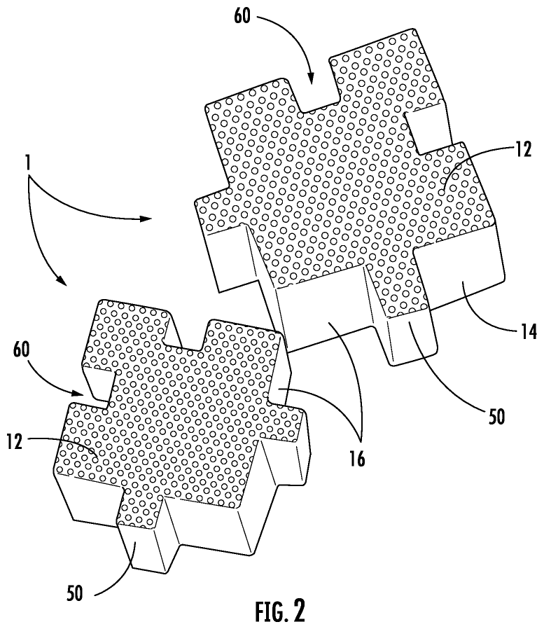

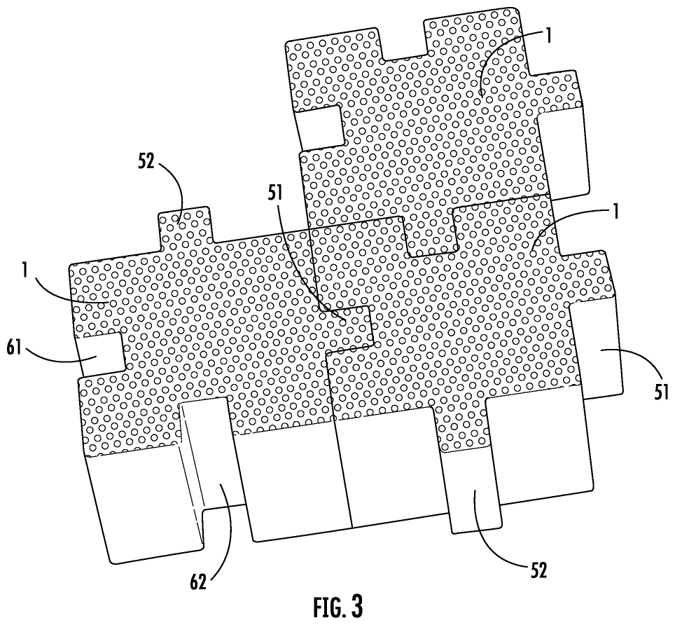

FIG. 2, for instance, illustrates two (2) separate IPHBs 1 in accordance with certain embodiments of the invention. Each of these IPHBs include a top surface 12, a bottom surface 14 and at least one side edge 16. The particular IPHBs 1 shown in FIG. 2 include at least one interlocking-male component 50 and at least one interlocking-female component 60. FIG. 3 illustrates three (3) interlocked IPHBs 1 in accordance with certain embodiments of the invention. The IPHBs 1 shown in FIG. 3 each include a first interlocking-male component 51, a second interlocking-male component 52, a first interlocking-female component 61, a second interlocking-female component 62.

FIG. 4 depicts top and side views of an IPHB 1 and illustrates the continuous polymeric matrix material 10 and the network of microporous channels and/or chambers 30 extending throughout the continuous polymeric matrix material, which are exposed at the surfaces of the IPHB, in accordance with certain embodiments of the invention. As illustrated by FIG. 4, a seeded IPHB may enable cells 33 to grow and migrate throughout the network of microporous channels and/or chambers 30 in a three-dimensional manner.

In accordance with certain embodiments of the invention, the at least one interlocking-male component includes a first interlocking-male component extending outwardly from the at least one side edge. For example, the at least one side edge may include a first side edge and a second side edge, in which the at least one interlocking-male component includes a first interlocking-male component extending outwardly from the first side edge and a second interlocking-male component extending outwardly from the second side edge. The interlocking-male components expending outwardly from the side edges, for instance, are configured to interlock or join to corresponding interlocking-female components of other IPHBs to form an expanding continuous 3D scaffolding system with the interlocked or joined IPHBs expanding outwardly in an x-y plane. In accordance with certain embodiments of the invention, the at least one interlocking-male component may also include a third interlocking-male component extending outwardly from the top surface. In this regard, the interlocking-male components expending outwardly from the top surface, for instance, are configured to interlock or join to corresponding interlocking-female components located on a bottom surface of other IPHBs to form an expanding continuous 3D scaffolding system with the interlocked or joined IPHBs expanding in a z-direction that is perpendicular to the x-y plane. In accordance with certain embodiments of the invention, for example, a plurality of IPHBs may be interlocked or joined together in both the x-y plane and stacked upon themselves in the z-direction. As noted above, the at least one interlocking-female component may include a first interlocking-female component extending inwardly from the at least one side edge towards an interior portion of the 3D macrostructure. For example, the at least one side edge may include a third side edge and a fourth side edge, in which the at least one interlocking-female component includes a first interlocking-female component extending inwardly from the third side edge towards an interior portion of the 3D macrostructure and a second interlocking-female component extending inwardly from the fourth side edge towards an interior portion of the 3D macrostructure. In such example embodiments, the first side edge and the third side edge may define a first pair of opposing side edges, while the second side edge and the fourth side edge may define a second pair of opposing side edges. In accordance with certain embodiments of the invention and as noted above, at least one interlocking-female component may include a third interlocking-female component extending inwardly from the bottom surface towards an interior portion of the 3D macrostructure. In this regard, a plurality of IPHBs may be interlocked or joined together in both the x-y plane and stacked upon themselves in the z-direction.

In accordance with certain embodiments of the invention, the interlocking feature of the IPHBs enable the custom formation of continuous 3D scaffolds for the growth or a variety of cells and/or tissues, in which the number of particular cells being seeded and/or grown in the continuous 3D scaffold is not limited. Such flexibility in the relative positioning and interlocking of the different IPHBs enable the custom growth of multiple types of cells that may form complex interfaces between different types of cells. For example, a plurality of interlocked IPHBs defining a continuous 3D scaffold (e.g., continuous network microporous channels and/or chambers extending throughout the plurality of interlocked IPHBs) may include from 1 to 20 different cell and/or tissue types being grown simultaneously, such as at least about any of the following: 1, 2, 3, 4, 5, 6, 7, 8, 9, and 10 different cell and/or tissue types being grown simultaneously, and/or at most about any of the following: 20, 19, 18, 17, 16, 15, 14, 13, 12, 11, and 10 different cell and/or tissue types being grown simultaneously.

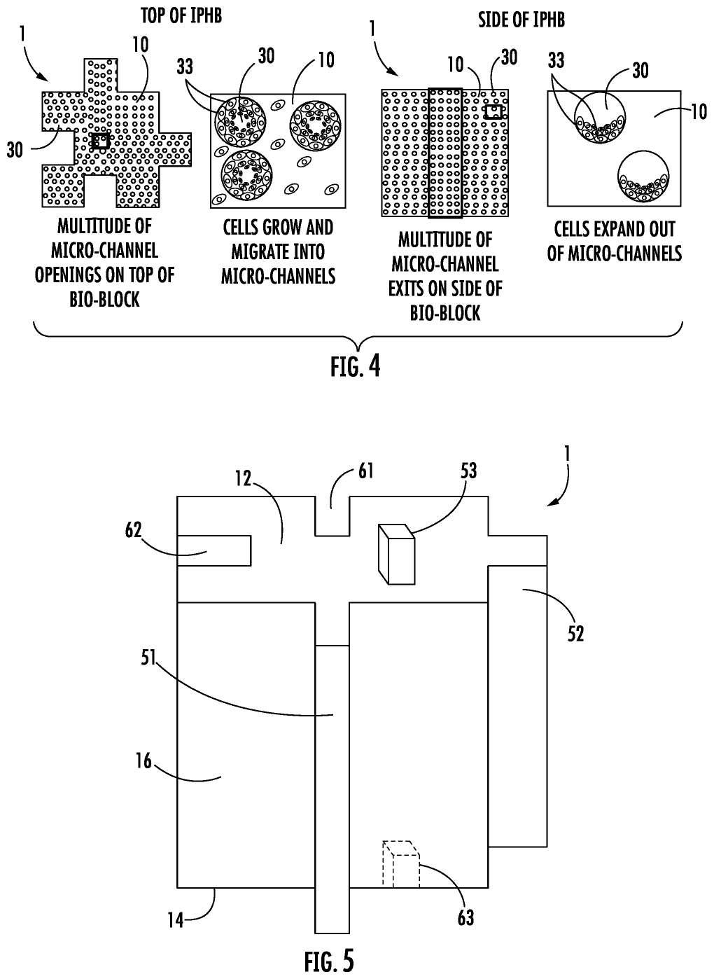

FIG. 5, for example, illustrates a IPHB 1 including an interlocking-male component 53 located on a top surface 12 and an interlocking-female component 63 protruding into the bottom surface 14 of the IPHB in accordance with certain embodiments of the invention. In this regard, the interlocking-male component 53 located on a top surface 12 and an interlocking-female component 63 protruding into the bottom surface 14 of the IPHB may be configured to engage and interlock with a separate IPHB as described herein.

In accordance with certain embodiments of the invention, the 3D macrostructure with the exception of the at least one interlocking-male component and at least one interlocking-female component may define a cube, a square prism, or a triangular prism. The 3D macrostructure with the exception of the at least one interlocking-male component and at least one interlocking-female component, in accordance with certain embodiments of the invention, define a polygonal prism having from 3 to 12 side edges, such as at least about 3, 4, 5, 6, 7, and 8 side edges, and/or at most about any of the following: 12, 11, 10, 9, and 8 side edges. In accordance with certain embodiments of the invention each side may include either an interlocking-male component and/or an interlocking-female component. Alternatively, some of the side edges may be devoid of an interlocking-male component and an interlocking-female component.

In accordance with certain embodiments of the invention, the at least one side edge includes a first side edge, a second side edge, and an arcuate side edge located between and adjacent the first side edge and the second side edge. For example, the first side edge may include the at least one interlocking-male component extending outwardly from the first side edge and the second side edge may include the at least one interlocking-female component extending inwardly from the second side edge towards an interior portion of the 3D macrostructure. In this regard, each IPHB may define a pie-like shape that when assembled or interlocked together forms a circle (e.g., cylinder since each IPHB has a thickness). Such configurations of the IPHBs may be desirable for use with circular culture wells. Additionally or alternatively, the at least one interlocking-male component includes a second interlocking-male component extending outwardly from the top surface, and/or the at least one interlocking-female component includes a second interlocking-female component extending inwardly from the bottom surface towards an interior portion of the 3D macrostructure. In this regard, a plurality of IPHBs may be stacked upon each other in a z-direction to form a thicker cylinder or semi-cylinder. In accordance with certain embodiments of the invention, the 3D macrostructure with the exception of the at least one interlocking-male component and at least one interlocking-female component may define a semi-cylinder, such as ⅛th of a cylinder to ½ of a cylinder, such as ⅛th, ¼th, ⅓rd, or ½ of a cylinder.

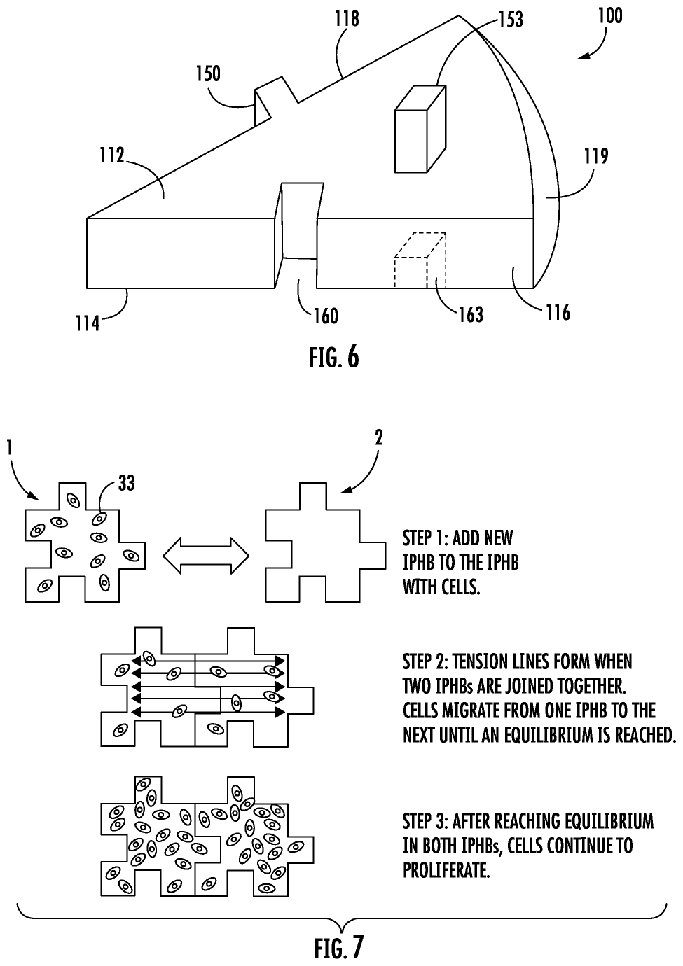

FIG. 6, for example, illustrates a semi-cylinder shaped IPHB 100 including an interlocking-male component 153 located on a top surface 112 and an interlocking-female component 163 protruding into the bottom surface 114 of the IPHB in accordance with certain embodiments of the invention. The semi-cylinder shaped IPHB 100 illustrated by FIG. 6 includes a first interlocking-male component 150 extending from a second side edge 118 and a first interlocking-female component 160 protruding into a second side edge 116. The first side edge 116 and the second side edge 118 of the IPHB 100 shown in FIG. 6 are connected via an arcuate side edge 119 at one end of the IPHB. In this regard, the interlocking-male component 153 located on a top surface 112 and an interlocking-female component 163 protruding into the bottom surface 114 of the IPHB may be configured to engage and interlock with a separate IPHB as described herein.

In accordance with certain embodiments of the invention, the at least one interlocking-male component may occupy or overlap from about 5% to about 50% of the macroscopic surface area of the surface (e.g., side edge, top surface, or bottom surface) upon which it extends from, such as at about any of the following: 10, 15, 20, and 25%, and/or at most about any of the following: 50, 45, 40, 35, 30, and 25%. Additionally or alternatively, the at least one interlocking-female component may occupy or overlap from about 5% to about 50% of the macroscopic surface area of the surface (e.g., side edge, top surface, or bottom surface) upon which it penetrated into, such as at about any of the following: 10, 15, 20, and 25%, and/or at most about any of the following: 50, 45, 40, 35, 30, and 25%.

In accordance with certain embodiments of the invention, the bottom surface may have a rougher texture relative to the top surface. For example, the top surface may be relatively smooth relative to the bottom surface which may have a textured structure. The textured structure at the bottom surface, for example, may facilitate the flow of a culture medium or washing medium through the entirety of the IPHB by providing structural spacers to facilitate the drainage of the culture medium or washing medium from the IPHB. The textured surface of the bottom surface, for example, may include a plurality of minor protrusions, such as individual nubs or ridges that function as short spacers. The plurality of minor protrusions, however, may be significantly smaller in size compared to the at least one interlocking-male component, such as being at most about 1/10th the size of the at least one interlocking-male component. In this regard, the minor protrusions may generally not provide any interlocking functionality in accordance with certain embodiments of the invention.

In accordance with certain embodiments of the invention, the top surface of the IPHB may comprise a macroscopic surface area from about 0.25 cm² to about 25 cm², such as at least about any of the following: 0.25, 0.5, 75, 1, 1.5, 2, 5, 8, 10, and 12 cm², and/or about any of the following: 25, 22, 20, 18, 15, and 12 cm². Additionally or alternatively, the bottom surface may comprise a macroscopic surface area from about 0.25 cm² to about 25 cm², such as at least about any of the following: 0.25, 0.5, 0.75, 1, 1.5, 2, 5, 8, 10, and 12 cm², and/or about any of the following: 25, 22, 20, 18, 15, and 12 cm². Additionally or alternatively, the thickness of the 3D macrostructure may be from about 0.5 cm to about 3 cm, such as at least about any of the following: 0.5, 0.75, 1, 1.25, and 1.5 cm, and/or at most about any of the following: 3, 2.5, 2, and 1.5 cm.

As noted above, the each of the at least one interlocking-female components may be configured to receive a corresponding at least one interlocking-male component of a second IPHB. In this regard, a plurality of IPHBs may be joined or interlocked together in an individually sequential addition to expand the 3D scaffold as desired along the x-y plane and alone the z-direction. For example, cell growth in a plurality of interconnected IPHBs may be continued in the z-direction by stacking layers of additional IPHBs on top of a first layer of IPHBs.

In accordance with certain embodiments of the invention, the continuous polymeric matrix material may be non-degradable. In this regard, the cells and/or tissue produced in the IPHB may need to be flushed out of the interior network of the network of microporous channels and/or chambers for further analysis, purification, or development. Additionally or alternatively, the continuous polymeric matrix material may be selectably degradable. For example, hydrogel formulations may be rendered biodegradable, such as by insertion of enzyme-sensitive sequences or utilization of native matrix-derived compounds. For example, the continuous polymeric matrix material may comprises a selectably degradable hydrogel material comprising one or more degradable polymers, such as one or more biopolymers derived from a living organism. The one or more biopolymers derived from a living organism, for example, may comprise a polynucleotide, polysaccharide, polypeptide, or any combination thereof. In accordance with certain embodiments of the invention, the one or more biopolymers may comprise collagen, gelatin, laminin, alginate, glycosaminoglycans, oligonucleotides (e.g., DNA, RNA), carbohydrates, lipids, cellulose, alginate, and proteins that can be gently and degraded, such as with the use of protein specific enzymes, ionic solvents, neutral detergents, weak acids, and peroxides to disrupt the biopolymer chains. In accordance with certain embodiments of the invention, the one or more biopolymers may comprise degradable monomers comprising esters, such as hydroxybutyrate, lactic acid, glycolic acid, and caprolactone; anhydrides, such as adipic acid, and sebacic acid; saccharides, such as cellulose, alginate, pectin, dextrin, chitosan, hyaluronan, Chondroitin sulfate, and heparin; proteins; nucleotides (DNA, RNA); peptides, such as collagen, gelatin, silk, and fibrin; urethanes; phosphates; carbonates; and vinyl chlorides. In accordance with certain embodiments of the invention, the selectably degradable hydrogel material may further comprise a synthetic polymer, such as a polyester, a polyanhydride, a polycarbonate, a polyurethane, a polyphosphate or combinations thereof. The continuous polymeric matrix material, in accordance with certain embodiments of the invention, may comprise a 3D cross-linked polymer network, a non-crosslinked polymer network, or a combination thereof.

The continuous polymeric matrix material, in accordance with certain embodiments of the invention, may comprise a swellable hydrogel material. The swellable hydrogel material may comprise a radically mediated reaction product of at least a first monomer including an acrylate or methacrylate functional groups and a second monomer or oligomer including at least two (2) free-radically polymerizable functional groups. For example, the at least two (2) free-radically polymerizable functional groups may independently from each other comprise an acrylate or methacrylate group, an allylic group, an alkynyl, a vinyl nitrile, a vinyl ether, a vinyl ester, a vinyl amide, a styrenic group, a maleate group, a fumarate group, or a norbornene group. In accordance with certain embodiments of the invention, at least one of the first monomer or the second monomer may comprise polyethylene glycol functionality (e.g., —O(C₂H₄O)ₙH; where n has a value from 1 to 100, polypropylene glycol functionality (e.g., —O(C₃H₆O)ₙH; where n has a value from 1 to 100, and/or glycerol functionality incorporated into a backbone of the monomer and/or grafted onto the monomer as a side-chain or a component of a side chain. By way of example only, the at least one of the first monomer or second monomer comprises 2-Hydroxyethyl acrylate (HEA), Poly(ethylene glycol)methyl ether acrylate (MPEGA), N-Methylacetamide (NMA), or Poly(ethylene glycol)diacrylate (PEGDA). In accordance with certain embodiments of the invention, non-limiting examples of non-degradable monomers that may be utilized in the hydrogel materials may include polyolefins (e.g., ethylene, propylene), styrene, nylon (e.g., amides), and/or acrylics. In accordance with certain embodiments of the invention, non-limiting examples of degradable monomers that may be utilized in the hydrogel materials may include esters (e.g., hydroxybutyrate, lactic acid, glycolic acid, caprolactone), anhydrides (e.g., adipic acid, sebacic acid) saccharides (e.g., cellulose, alginate, pectin, dextrin, chitosan, hyaluronan, Chondroitin sulfate, heparin), proteins, nucleotides (e.g., DNA, RNA), peptides (e.g., collagen, gelatin, silk, fibrin), urethanes, phosphates, carbonates, and vinyl chlorides. Additionally or alternatively, a third monomer comprising a cross-linking agent may incorporated continuous polymeric matrix material. Additionally or alternatively, the swellable hydrogel material may comprise one or more natural polymers, such as plant-derived polymers (e.g., cellulosic-polymers) and animal-derived polymers.

In accordance with certain embodiments of the invention, the continuous polymeric matrix material may mimic a natural tissue of interest by including one or more physical properties within about 20%, such as within about 15%, 10%, 8%, 5%, 3%, or 1%, of the natural tissue of interest, wherein the one or more physical property of interest includes softness and tension. For example, the one or more physical properties may comprise an elastic and/or compressive modulus, a storage modulus at 1 Hz, loss of modulus at 1 Hz, and/or protein/chemical coating (e.g., Collagen I, II, III, IV, Laminin I, II, Hyaluronan, Gelatin, Fibrin, Fibronectin, etc.). By way of example only, native adipose tissue has a storage modulus at 1 Hz from 50-100 kPa, a loss of modulus at 1 Hz of 10-20 kPa, and an elastic and/or compressive modulus of 3 kPa. In this regard, for example, a IPHB may have a storage modulus at 1 Hz of about 110 kPa, a loss of modulus at 1 Hz of about 22 kPa, and an elastic and/or compressive modulus of about 3 kPa. The IPHB, for example, may be analyzed with a Dynamic Mechanical Analyzer (DMA; TA Instruments, RSA3) setup at to assess mechanical and physical properties. A 5-mm biopsy punch may be used to isolate a circular hydrogel sample to prevent force-concentrating points. DMA may be performed via a dynamic cylindrical compression analysis with a rate of compression of 0.005-mm/sec and a frequency sweep at one 1-Hz. For example, the particular chemical constituents and/or degree of crosslinking may be altered to tailor one or more physical and/or mechanical properties of the resulting continuous polymeric matrix material to mimic or mirror those associated with a natural tissue of interest. Additionally or alternatively, the surface topography/texture of the network of microporous channels and/or chambers and/or the outside of the IPHBs can manipulated. Most of these surfaces may be smooth, grooves, bumps, mounds, divots, and other surface irregularities may be introduced to alter the flow of liquid or gas through the network of microporous channels and/or chambers. Such surface irregularities, for example, may introduce turbulence to help slow the flow of liquids or gases throughout the network of microporous channels and/or chambers. By way of example only, the surface irregularities may be significantly smaller in size compared to the average diameter of the network of microporous channels and/or chambers, such as being at most about ¼th to about 1/10th the size of the average diameter of the network of microporous channels and/or chambers.

In accordance with certain embodiments of the invention, the continuous polymeric matrix material is formed via an additive manufacturing technique, such as 3D printing or digital light synthesis printing. In this regard, the network of microporous channels and/or chambers is structured to mimic the morphology of a natural tissue of interest, such as by varying the geometry and dimensions of the network of microporous channels and/or chambers to mirror the morphology of the natural tissue of interest. For instance, the morphology of a natural tissue of interest may be readily ascertained by one of skill in the art, and this morphology may be duplicated via a 3D printing or digital light synthesis printing operation to form an IPHB having a network of microporous channels and/or chambers that mimics the morphology of the natural tissue of interest.

In accordance with certain embodiments of the invention, the average diameter of the network of microporous channels and/or chambers may comprise from about 100 to about 800 microns, such as at least about any of the following: 100, 120, 150, 180, 200, 220, and 250 microns, and/or at most about any of the following: 800, 780, 750, 720, 700, 680, 650, 620, 600, 580, 550, 520, 500, 480, 450, 420, 400, 380, 350, 320, 300, 280, and 250 microns. Additionally or alternatively, the network of microporous channels and/or chambers may comprise at least about 40% by volume of the 3D macrostructure, such as from at least about any of the following: 40, 50, 60, and 70% by volume of the 3D macrostructure, and/or at most about any of the following: 90, 85, 80, 75, and 70% by volume of the 3D macrostructure.

In accordance with certain embodiments of the invention, an interface between the network of microporous channels and/or chambers and continuous polymeric matrix material may comprises a coating of a compatibilizer selected to promote adhesion of a primary cell of interest. This coating may be applied subsequent to formation of the IPHB. By way of example, the coating comprising the compatibilizer may comprise a biological coating including, for example, Collagen I (e.g., Human Mesenchymal Stem Cells [from Adipose, Bone Marrow, Umbilical Cord], Human Neonatal Dermal Fibroblasts, Human Adult Dermal Fibroblasts, Human Keratinocytes, Human Myocytes, Human Osteoblasts, Human Osteocytes, Human Chondrocytes, Bovine Myocytes, Porcine Hepatocytes, Porcine Chondrocytes, Porcine Osteocytes, Equine Muscle Derived Stem Cells); Laminin I (e.g., Human Induced Pluripotent Stem Cells, Mouse Dorsal Root Ganglia); Hyaluronan (e.g., Porcine Hepatocytes, Human Dermal Adult Fibroblasts); Gelatin (e.g., Human Mesenchymal Stem Cells [from Adipose, Bone Marrow, Umbilical Cord], Human Neonatal Dermal Fibroblasts, Human Adult Dermal Fibroblasts, Human Keratinocytes, Human Myocytes, Human Osteoblasts, Human Osteocytes, Human Chondrocytes, Human T cells (CD8+), Human T cells (CD4+), Human Macrophages, Bovine Myocytes, Porcine Hepatocytes, Porcine Chondrocytes, Porcine Osteocytes, Equine Muscle Derived Stem Cells); Fibrin (e.g., Human Keratinocytes); Fibronectin (e.g., human Mesenchymal Stem Cells [from Adipose, Bone Marrow, Umbilical Cord], Human Neonatal Dermal Fibroblasts, Human Adult Dermal Fibroblasts, Human Keratinocytes, Human Osteoblasts, Human Osteocytes, Human Chondrocytes); or any combinations thereof.

In another aspect, the present invention provides a scaffolding system comprising a plurality of IPHBs, such as those described and disclosed herein. In accordance with certain embodiments of the invention, for instance, the plurality of IPHBs includes a first IPHB including a first interlocking-male component and a second IPHB including a second interlocking-female component, in which the second interlocking-female component is configured to receive the first interlocking-male component. In accordance with certain embodiments of the invention, for example, the first interlocking-male component may be inserted into the second interlocking-female component, in which the first IPHB and the second IPHB are each provided in a swollen state thereby improving interlocking of the first IPHB and the second IPHB. In this regard, the swollen state may be provided due to the absorbance of a liquid, such as water or a culture medium. As the first and second IPHBs swell during an interlocked state, the frictional forces between the mated interlocking-male and interlocking-female component(s) increases to increase the force required to separate the IPHBs.

In accordance with certain embodiments of the invention, the first IPHB has a first network of microporous channels and/or chambers and the second IPHB has a second network of microporous channels and/or chambers, in which a first portion of the first network of microporous channels and/or chambers at least partially overlaps with a first portion of the second network of microporous channels and/or chambers when the first IPHB and the second IPHB are interlocked and define an aggregate continuous network of microporous channels and/or chambers. In this regard, the density of microporous channels at the surfaces of the IPHBs is sufficiently large, as noted above, such that partial overlap of a least portions of the microporous channels at the surfaces of the respective IPHBs enables formation of a continuous aggregate continuous network of microporous channels and/or chambers extending throughout the each IPHB that may be interlocked. As noted above, a plurality of IPHBs may be interlocked together along that x-y plane and/or along the z-direction.

In accordance with certain embodiments of the invention, the first IPHB may be seeded with a first primary cell and the second IPHB is seeded with a second primary cell, wherein the first primary cell is different than the second primary cell. As noted above, each of the IPHBs may be seeded by a different and/or unique primary cell or a combination of a plurality of primary cells. Alternatively, for mass production of a given cell of interest, each IPHB may be seeded with the same primary cell.

By way of example only, one or more of the IPHBs may be seeded and enable cell growth of any of the following: (1) Human Stem Cells, such as Human Wharton's Jelly Cells (MSC), Human Bone Marrow Derived Mesenchymal Stem Cells (MSC), Human Adipose Derived Mesenchymal Stem Cells (MSC), Human Skin Derived Induced Pluripotent Stem Cells (iPSC), Human Blood Cell Derived Induced Pluripotent Stem Cells (iPSCs), Human CD4+ T Cells, Human CD8+ T Cells; (2) Primary Mammalian Cells, such as HepG2 Cells (Liver Carcinoma Cells), Human Adult Dermal Fibroblasts (Primary Cells), Human Neonatal Dermal Fibroblasts (Primary Cells), Human Adult Keratinocytes (Primary Cells), Mouse Dorsal Root Ganglia (Primary Neural Cells), Bovine Myocytes (Primary Cell Line), Primary Porcine Hepatocytes, Porcine Chondrocytes, Porcine Osteocytes, Equine Muscle Derived Stem Cells (Primary MSCs), Primary Snail Cells, Human Macrophages; (3) Immortalized Mammalian Cell Lines, such as UB-OC2 Cells (Mouse Cochlear Epithelium), Human Myoblastoma (Muscle Tumor), PC3 (Prostate Cancer), CHO (Chinese Hamster Ovary Cell), HEK293 (Human Embryonic Kidney Cell), SHSY5Y (Neuronal Tumor), PANC-1 (Human Pancreatic Cancer), HeLa (Cervical Cancer), A549 (Lung Cancer), A673 (Muscle Cancer); and (4) Primary Plant Cells, such as Rosemary, Tobacco, and Tomato.

FIG. 7 illustrates cell 33 migration from a first IPHB 1 to a second IPHB 2 when interlocked together in accordance with certain embodiments of the invention. As illustrated in FIG. 7 the first IPHB 1 may be seeded with cells 33 and then joined to an empty (e.g., devoid of cells) second IPHB 2. Once joined together, the cells 33 migrate from the first IPHB 1 into the second IPHB 2 until an equilibrium is reached. After reaching equilibrium in both IPHBs, the cells 33 will continue to proliferate.

As noted above, the methods of cultivating cells in accordance with certain embodiments of the invention utilize one or more IPHB(s). By way of example, for instance, the method of cultivating cells may comprise providing an initial scaffolding comprising a first IPHB and seeding the first IPHB with one or more cells of interest (e.g., a first cell type). Once seeded, the one or more cells may be provided appropriate nutrition via one or more culture medias, which may reach the seeded cells via the microporous network formed in the first IPHB. In this regard, old or exhausted culture media may be flushed out and/or sucked out of the IPHB and/or the well that the IPHB may be housed within. After removal of old or exhausted culture media, fresh culture media may be applied to the IPHB. For instance, a first culture media may be desired at the outset of growth upon seeding, but a modified (different) culture media may be preferred once cell growth is well established and propagation of the cells through the IPHB is well established. In this regard, the method may comprise feeding the one or more cells of interest to facilitate or allow the one or more cells of interest to propagate throughout the network of microporous channels and/or chambers. As the cells grow and propagate throughout the first IPHB, the initial scaffolding may be expanded by interlocking a second IPHB to the first IPHB. In this regard, the first IPHB has a first network of microporous channels and/or chambers and the second IPHB has a second network of microporous channels and/or chambers that at least partially overlap at an interface between the first IPHB and the second IPHB. In this regard, the one or more cells of interest are allowed to propagate from the first IPHB into the second IPHB, where culture media may be provided to facilitate the growth and propagation of the cell line throughout the second IPHB.

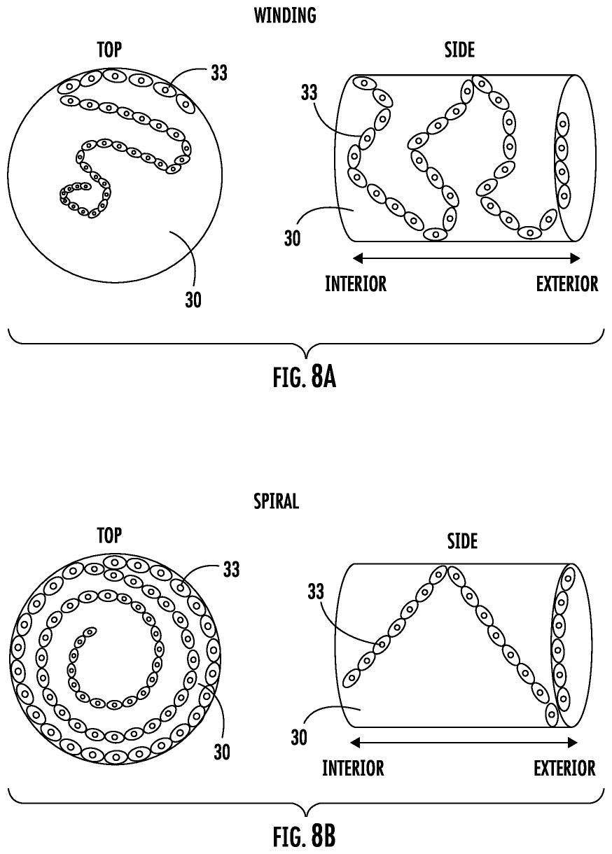

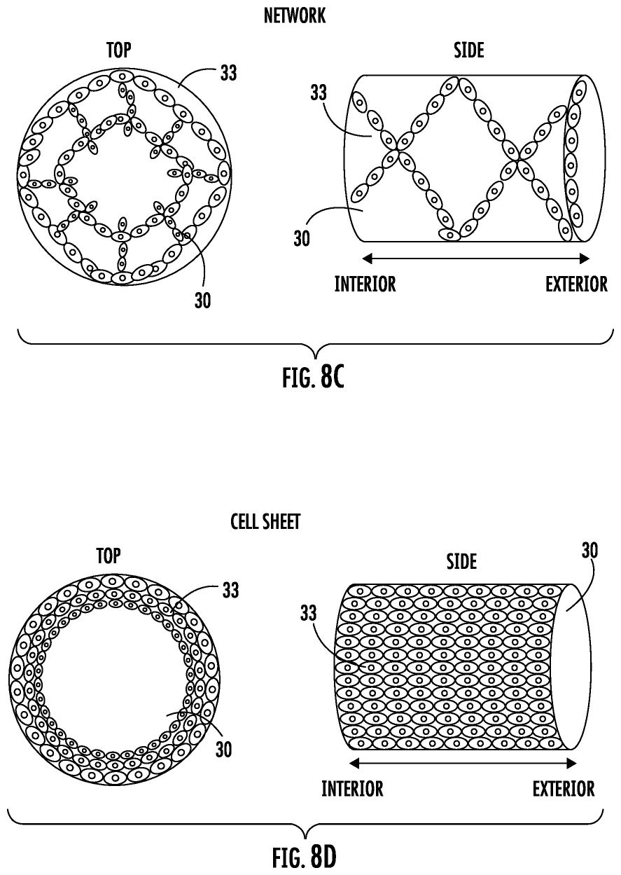

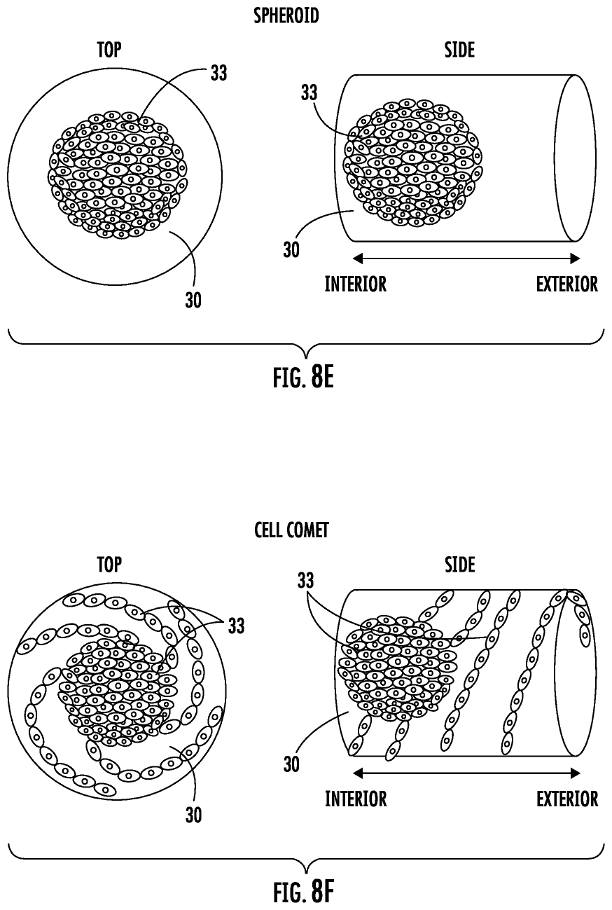

In accordance with certain embodiments of the invention, the one or more cells of interest that have been seeded into the network microporous channels and/or chambers may propagate within the network in a variety of patterns. As illustrated in FIG. 8A, for instance, the one or more cells of interest 33 (e.g., seeded in an IPHB) grow and propagate through the network of microporous channels and/or chambers 30 and define a winding pattern wherein the one or more cells of interest wind back-and-forth in a 3D configuration across at least a portion of individual microporous channels as the one or more cells of interest propagate from an interior portion of the first IPHB towards one or more exterior surfaces, such as the top surface, the bottom surface, and/or or the at least one side edge. As illustrated in FIG. 8B, for instance, the one or more cells of interest 33 (e.g., seeded in an IPHB) grow and propagate through the network of microporous channels and/or chambers 30 and define a spiral pattern adhered to walls of the network of microporous channels and/or chambers as the one or more cells of interest propagate from an interior portion of the first IPHB towards one or more exterior surfaces, such as the top surface, the bottom surface, and/or or the at least one side edge. As illustrated in FIG. 8C, for instance, the one or more cells of interest 33 (e.g., seeded in an IPHB) grow and propagate through the network of microporous channels and/or chambers 30 and define a network of nodes and branches defining open regions therebetween as the one or more cells of interest propagate from an interior portion of the first IPHB towards one or more exterior surfaces, such as the top surface, the bottom surface, and/or or the at least one side edge. As illustrated in FIG. 8D, for instance, the one or more cells of interest 33 (e.g., seeded in an IPHB) grow and propagate through the network of microporous channels and/or chambers 30 and define a continuous cell sheet adhered to walls of the network of microporous channels and/or chambers as the one or more cells of interest propagate from an interior portion of the first IPHB towards one or more exterior surfaces, such as the top surface, the bottom surface, and/or or the at least one side edge. As illustrated in FIG. 8E, for instance, the one or more cells of interest 33 (e.g., seeded in an IPHB) grow and propagate through the network of microporous channels and/or chambers 30 and define a spheroid. As illustrated in FIG. 8F, for instance, the one or more cells of interest 33 (e.g., seeded in an IPHB) grow and propagate through the network of microporous channels and/or chambers 30 and define a comet-like structure having a spheroid structure and a spiral structure emanating from the spheroid structure as the one or more cells of interest propagate from an interior portion of the first IPHB towards one or more exterior surfaces, such as the top surface, the bottom surface, and/or or the at least one side edge.

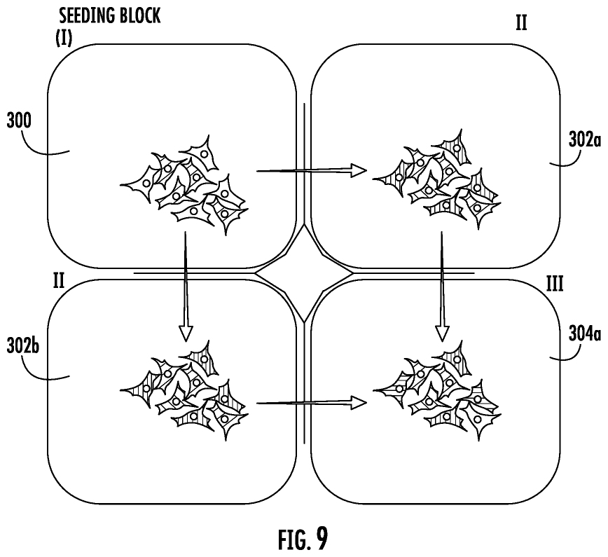

As noted above, the methods in accordance with certain embodiments of the invention enable 3D cell growth and propagation formations that uniquely resemble in vivo growth and propagation. In this regard, FIG. 9 is a schematic illustrating the nomenclature and conceptual cell migration of a plurality of IPHBs in accordance with certain embodiments of the invention. As shown in FIG. 9, an initially seeded IPHB 300 may be referenced as the prime or seed block (I). Once the seed block 300 reaches the desired confluency, which is cell phenotype dependent and may take from one to two weeks, it can be expanded by interlocking, for example, two (2) secondary (II) IPHBs 302a, 302b directly to the seed block 300. The number of secondary, tertiary, etc. may depend on the ultimate goal for cell and/or tissue growth. FIG. 9 illustrates a single tertiary (III) IPHB directly 304a attached to both of the secondary (II) 302a, 302b IPHBs. FIG. 10 is a schematic illustrating additional options for adding a varying number and positioning of additional IPHBs to those of FIG. 9. FIG. 10 illustrates three (3) example additions including a variety of configurations including one example having a; quaternary (IV) IPHB 308. As illustrated by FIGS. 9-10, the methods utilizing a plurality of IPHBs provide a myriad of different continuous growth scaffolds for customizing a growth protocol.

In accordance with certain embodiments of the invention, the method comprises a chain-cultivation method comprising the sequential addition of a plurality of secondary blocks to the first IPHB. The plurality of secondary blocks may include the second IPHB and a third IPHB interconnected directly to the second IPHB such that the second IPHB is located directly between the first IPHB and the third IPHB, and wherein the plurality of secondary IPHBs are initially devoid of cells. In this regard, the cells of interest located in the first IPHB are harvested after cell propagation from the first IPHB to the second IPHB, and the cells of interest located in the second IPHB are harvested after cell propagation from the second IPHB to the third IPHB. Such methods, therefore, enable the continual growth and harvesting of the cells of interest, particularly without the need for re-seeding or sub-culturing. In accordance with certain embodiments of the invention, the number of secondary blocks (i.e., secondary IPHBs) may be from at least 2, such as at least about any of the following: 2, 3, 5, 10, 20, 30, 40, 50, 60, 70, 80, 90, and 100 blocks.

In accordance with certain embodiments of the invention, the method comprises a multi-cell cultivation method, in which the one or more cells of interest seeded in the first IPHB comprises a first cell type and the second IPHB is seeded with a second cell type. In this regard, the first cell type is different than the second cell type. In this regard, the cells of the first cell type (e.g., bone cells) and cells of the second cell type (e.g., cartilage cells) are allowed to propagate towards each other and form a first interface between the first cell type and the second cell type. In accordance with certain embodiments of the invention, the method may further comprise a step of degrading each of the IPHBs to expose each cell type and the first interface. In this regard, the each cell type and the first interface may be isolated for further analysis and/or use. In accordance with certain embodiments of the invention, the method may further comprise interlocking a third IPHB directly together with the second IPHB, wherein the second IPHB is located directly between the first IPHB and the third IPHB, and seeding the third IPHB with a third cell type that is different from the first cell type and the second cell type. The method may also comprise allowing and/or growing the cells of the second cell type and cells of the third cell type to propagate towards each other and form a second interface between the second cell type and the third cell type. In accordance with certain embodiments of the invention, the method may further comprise a step of degrading each of the IPHBs to expose each cell type, the first interface, and the second interface.

As noted above, the one or more cells of interest may produce or secrete a therapeutic agent of interest, such as a biologic. For example, the therapeutic agent may comprise exosomes, extracellular vesicles, growth factors, monoclonal antibodies, peptides, proteins, viral particles, oligonucleotides, organelles, or combinations thereof. In this regard, various configurations of multiple IPHBs may be interlocked to provide an aggregate scaffolding network, in which multiple cell lines, such as those that may produce or secrete a therapeutic agent, are seeded in different IPHBs.

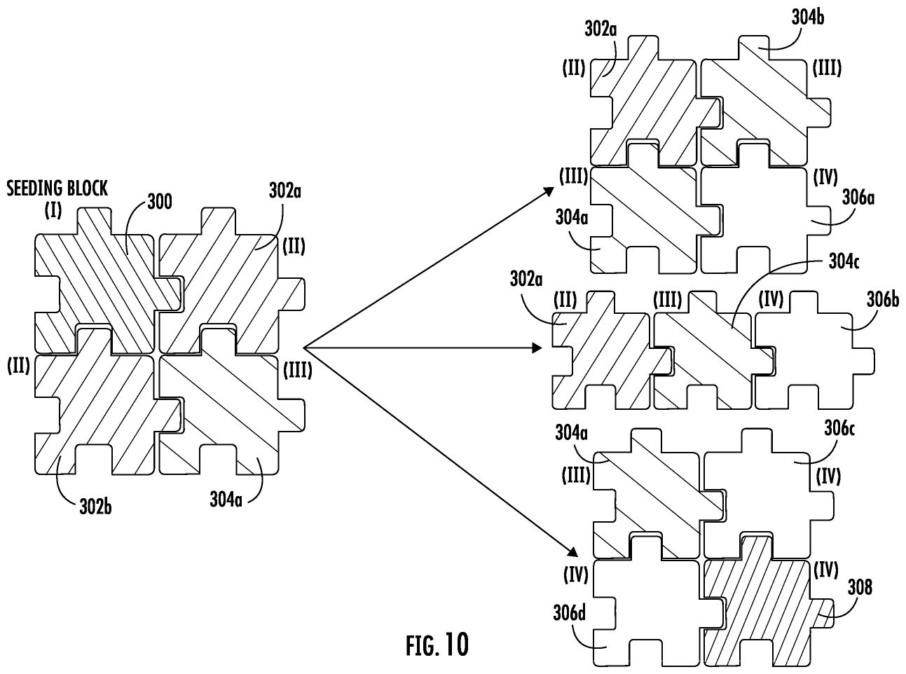

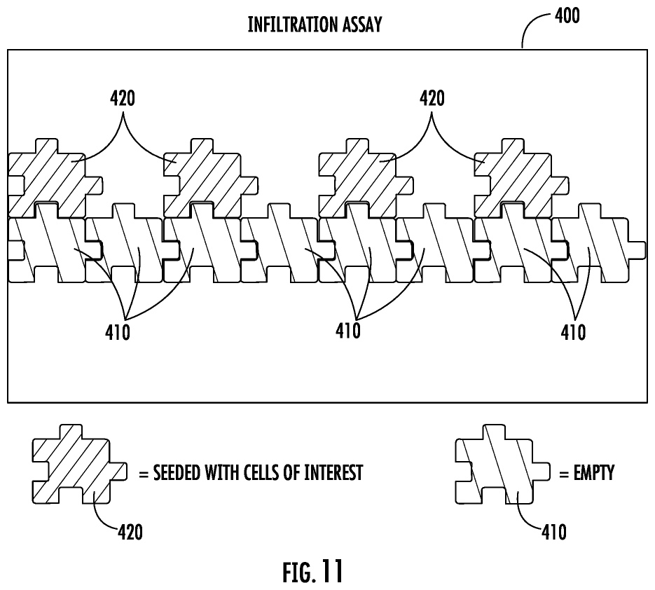

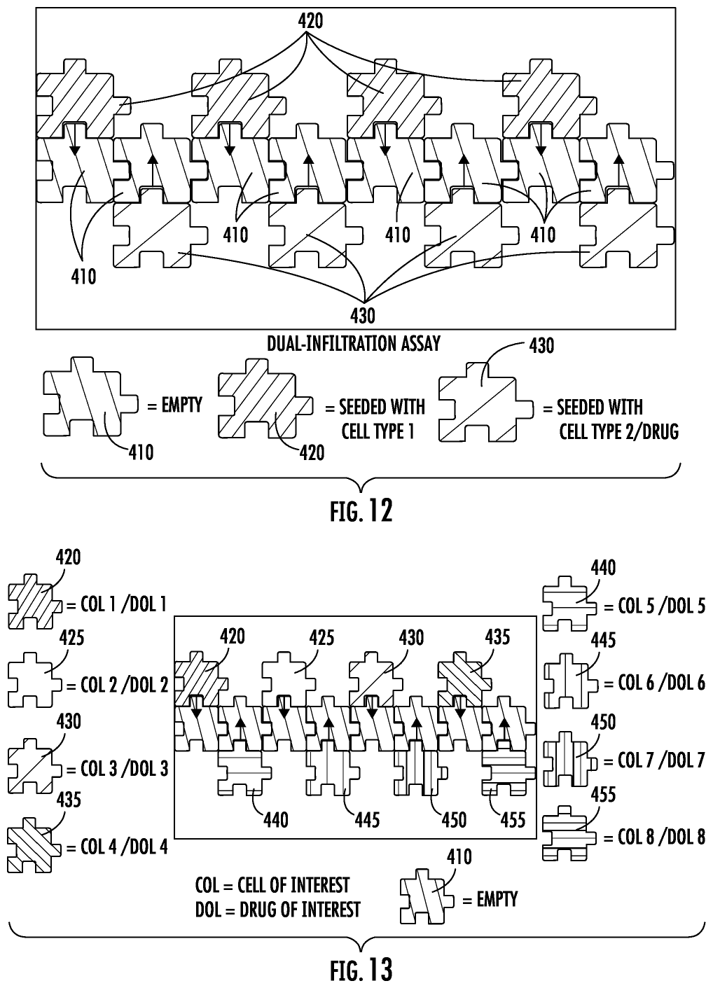

As noted above, the methods in accordance with certain embodiments of the invention may include interlocking a plurality of IPHBs in a myriad of different configurations to provide a wide variety of continuous growth scaffolds for customizing a growth protocol for a wide variety of different cell lines. FIGS. 11-21 illustrate just a few non-limiting example configurations in accordance with certain embodiments of the invention. FIG. 11, for instance, is a schematic for an infiltration assay configuration of a series of interlocked empty IPHBs 410 (e.g., IPHBs that are initially devoid of seeded cells) with four (4) seeded IPHBs 420 interlocked as side-chains to the series of interlocked empty IPHBs. All of the IPHBs are housed within a tray for housing the IPHBs and/or cell culture media. In this regard, the seeded cells in the seeded IPHBs 420 are grown and propagate into the series of interconnected empty IPHBs. FIG. 12 is a schematic for a dual-infiltration assay configuration of a series of interlocked empty IPHBs 410 with a first set of four (4) IPHBs seeded with a first cell type 420 interlocked as side-chains to the series of interlocked empty IPHBs and a second set of four (4) IPHBs seeded with a second cell type 430, which may produce or secrete a therapeutic agent interlocked as side-chains on an opposite side of the series of interlocked empty IPHBs. In this regard, the series of initially interlocked empty IPHBs 410 (e.g., devoid of seeded cells) can be penetrated simultaneously from cell lines from IPHBs 420 and IPHBs 430. In this particular configuration, the first and second cell types may form a plurality in interfaces and/or regions of interaction with each other in the initially empty IPHBs 410. FIG. 13, for example, illustrates a multi-infiltration assay configuration in which eight IPHBs 420, 425, 430, 435, 440, 445, 450, 455 are attached as side-chains to a series of interlocked empty IPHBs 410, in which each IFPB attached as a side-chain is seeded with a different cell type, each of which may produce or secret a different therapeutic agent. In this regard, the growth and propagation of multiple cell lines may be simultaneously grown and propagated into the series of interlocked initially empty IPHBs 410. In this regard, a plurality of different interfaces between the different cell types or cell lines may also be formed throughout the series of interlocked initially empty IPHBs.

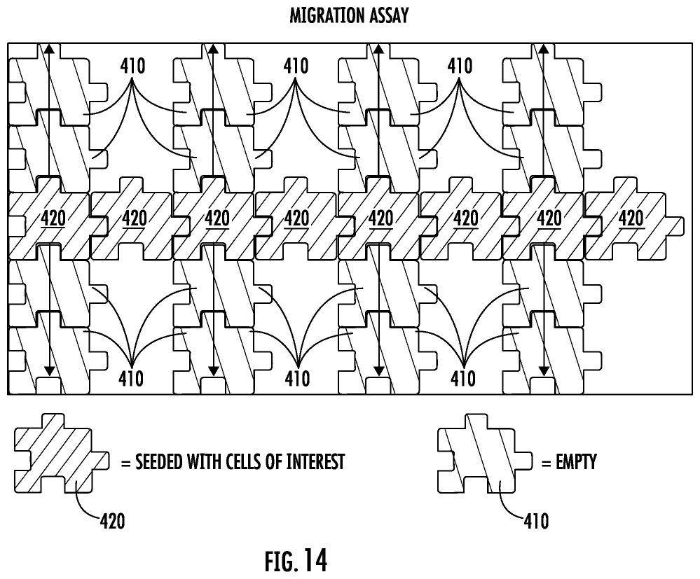

FIG. 14 illustrates a migration assay configuration in which a series of seeded IPHBs 420 have eight (8) side-chains of unseeded or initially empty IPHBs 410 attached thereto. Such a configuration, for example, may provide a multi-chain culturing method, such as noted above. That is, the cells of interest seeded in IPHBs 420 are grown and propagate outwardly through each of the initially empty IPHBs 410, in which earlier IPHBs penetrated by cell growth (those closest to the seeded IPHBs 420) can be harvested while the cell line growth is simultaneously grown in the subsequent IPHBs (e.g., those farther away from the seeded IPHBs 420). Such an approach, for instance, may provide a mass production of cells of interest in which cells may be harvested without ceasing the continued 3D growth of the cells of interest.

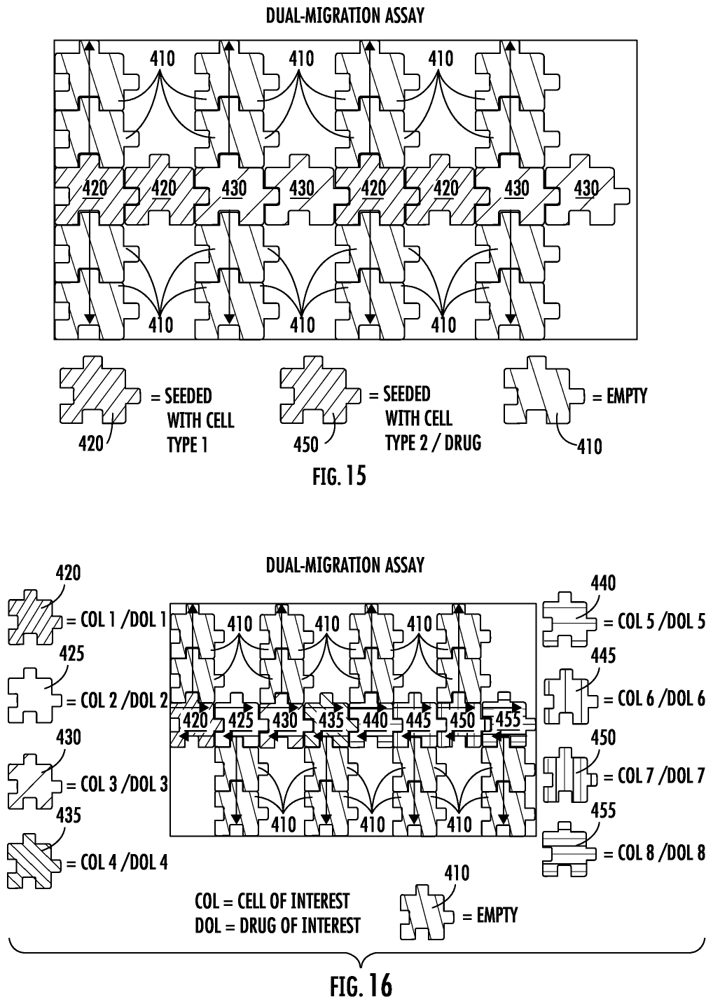

FIG. 15 illustrates a dual-migration assay configuration in which a series of seeded IPHBs include a first group of IPHBs are seeded with a first cell type 420 and a second group of IPHBs are seeded with a second cell type 430, in which eight (8) side-chains of unseeded or initially empty IPHBs 410 are attached thereto. In this regard, such a configuration may provide a mass production of a plurality of different cell types of interest in which each type of cells may be harvested without ceasing the continued 3D growth of the plurality of different cells of interest. FIG. 16 illustrates a multi-migration assay configuration in which a series of seeded IPHBs 420, 425, 430, 435, 440, 445, 450, 455, in which each IPHB is seeded with a different cell type, and eight (8) side-chains of unseeded or initially empty IPHBs 410 are attached thereto. As shown in FIG. 16, each of the different cell types may produce or secrete a therapeutic agent of interest.

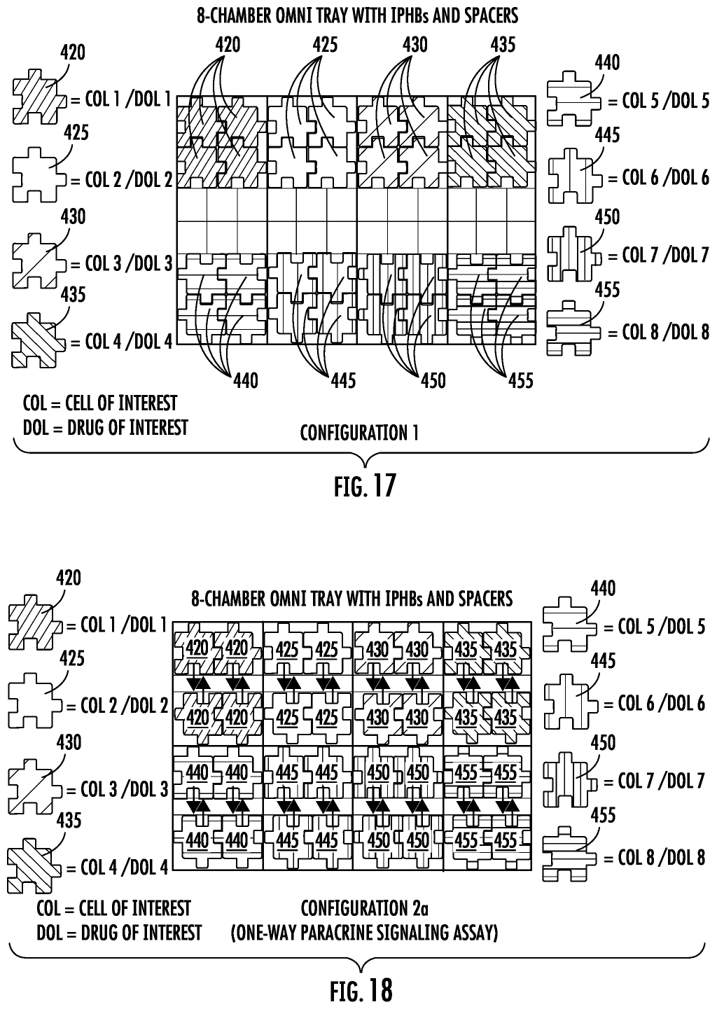

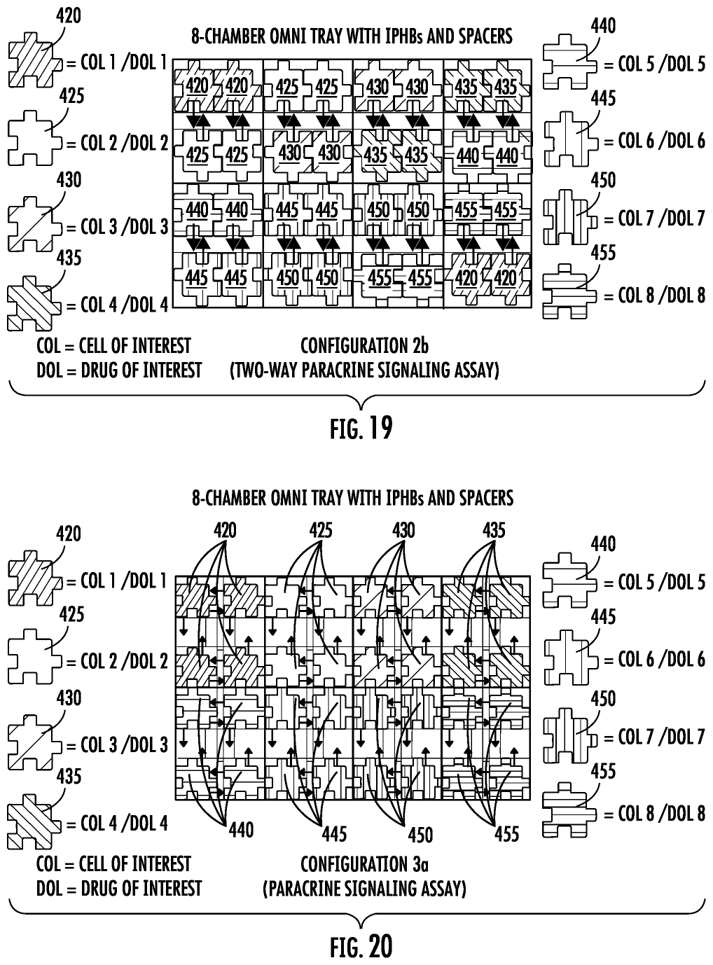

FIG. 17 illustrates an 8-chamber omni tray filled with a plurality of IPHBs 420, 425, 430, 435, 440, 445, 450, 455, and spacers, in which each chamber is filled with a group of four (4) interlocked IPHBs seeded with a different cell type (e.g., each chamber has its own cell type therein). Such a configuration, enables the production of multiple cell lines, which may produce or secrete a therapeutic of interest for isolation, in the same tray over the same period of time. FIG. 18 illustrates an 8-chamber omni tray filled with a plurality of IPHBs and spacers, in which the IPHBs are configured as a one-way paracrine signaling assay. FIG. 19 illustrates an 8-chamber omni tray filled with a plurality of IPHBs and spacers, in which the IPHBs are configured as a two-way paracrine signaling assay. FIG. 20 illustrates an 8-chamber omni tray filled with a plurality of IPHBs and spacers, in which the IPHBs are configured as paracrine signaling assay. FIG. 21 illustrates an 8-chamber omni tray filled with a plurality of IPHBs and spacers, in which the IPHBs are configured as a four-way paracrine signaling assay.

The foregoing example configurations are merely illustrative of a few possible methods according to certain embodiments of the invention. That is, the foregoing example configurations and figures are merely illustrative and non-limiting.

### EXAMPLES

The present disclosure is further illustrated by the following examples, which in no way should be construed as being limiting. That is, the specific features described in the following examples are merely illustrative and not limiting.

In this example, a 3D hydrogel system is demonstrated that improves expansion outcomes of MSC populations, such as adipose-derived MSCs (ASCs). The customizable 3D hydrogel system was bioprinted and was formulated to be a bioinert substrate that closely mimicked native adipose tissue mechanics and ultimately acts like a tailored bioreactor for MSC expansion and collection of biologics. The 3D hydrogel system was constructed with a unique ‘puzzle-piece’ macrostructure design (e.g., a plurality IPHBs) that enables easy addition of supplementary hydrogels (e.g., a IPHB). Additionally, the unique porous microarchitectural design permits mass transport and promotes cellular migration and proliferation, eliminating the need to subculture cells via cellular migration between hydrogels within the microchannels. The initial utilization of a bioinert substrate allowed for the investigation and observation of the potential role of the mechanical and dimensional properties of the 3D system on ASC senescence and stem-like phenotypic properties without introducing a bioactive substrate. It was believed that the softer, 3D hydrogel substrate would provide a more natural mechanical environment for the ASCs and result in the retention of nonsenescent stem-like ASC populations, relative to traditional 2D culture methodologies. Moreover, the unique architectural design would allow for continuous expansion of ASCs via cellular migration between the attached hydrogels (e.g., attached IPHBs), thus eliminating exposure of cells to negative 2D subculturing procedures and subsequent sequelae.

### Materials & Methods

### Cell Culture

Human ASCs (Lot #18TL212639, 23-year-old female, Black), human keratinocytes (KCs; Lot #18TL318559, 62-year-old male, Caucasian) obtained from Lonza (Basel, Switzerland) were utilized for this study. MSC-GM Mesenchymal Stem Cell Growth Medium BulletKit™ was obtained from Lonza (#PT-3001) and used for ASCs.

MesenCult™-ACF Plus Medium Kit (Stem Cell Technologies, BC, Canada; Cat. #05445) was used as the serum-free medium. DermaLife K Keratinocyte Medium Complete Kit was obtained from Lifeline Cell Technologies (MD, USA; #LL-0007) and used for KCs.

### 3D-Printed Hydrogel Cell Culture System

The bioinert 3D hydrogel system (e.g., IPHB) was approximately 1×1×1 cm and is a polyethylene glycol (PEG)-based system containing a unique microarchitectural design and was fabricated to resemble the mechanics of native adipose soft tissue and demonstrated no significant changes in mechanical properties over 3 months. The 3D hydrogels were placed into a glass six-well culture plate for culturing. Fibronectin is a commonly selected coating substrate for ASCs due to their natural secretion of fibronectin. Because the cells do not efficiently adhere/attach to the PEG-based polymer, both the 2D culture plastic/glass and 3D hydrogel were coated with fibronectin at a concentration of 5 μg/cm² to enhance the initial cell attachment. The concentration of fibronectin was standardized to surface area due to the inherent surface-area-to-volume differences between 2D and 3D systems. The approximate surface area of the 3D hydrogel was calculated from the 3D model used for bioprinting.

### Expansion of ASCs & KCs

ASCs and KCs were seeded within a T-150 flask and cultured until ˜80% confluence before subculturing (passaging). Subculturing of cells was performed by removing culture media, washing thrice with Hanks' balanced salt solution (HBSS, MA, USA; calcium-free, magnesium-free) and incubating with 0.05% Trypsin/EDTA (Lonza; Cat. #CC-3232) at 37°° C. for 5 min. Trypsin was neutralized with serum and the cells were centrifuged at 500×g for 5 min, then pelleted and resuspended for reseeding on new 2D tissue culture plastic vessels. ASCs at passage 1 (P1) were reseeded onto 2D culture plastic or onto/within the bioprinted 3D hydrogel (e.g., IPHB) system via dropwise addition of a concentrated cell solution to the surface of the hydrogels. This process was repeated with the residual cell solution five times to ensure efficient cell seeding. This repetitive seeding process allowed for the cells to distribute throughout the microporous structure within the hydrogel system. Given that the increased surface area and attachment of additional hydrogels eliminated the need for subculturing for this study, a passage-equivalence time point was utilized to allow for analogous comparison with 2D culture. Thus, a passaging event typically occurred every 4-5 days in 2D culture for ASCs but not in 3D culture. After 4-5 days of 2D culture for P2 ASCs, the cells were then subcultured and considered to be P3 in 2D, and the 3D ASCs were then considered P3 passage-equivalent. Culture expansion was determined based on the known 2D and 3D surface areas, initial cell seeding density and average population doubling time of 2.25 days (experimentally determined in 2D; data not shown) for the ASCs in order to standardize cell numbers. ASCs were seeded at a standardized concentration of 5000 cells/cm² for assays. Media supplementation was standardized to 150 μl/cm² for ASC expansion to account for dilutional differences in surface-area-to-volume ratio between 2D and 3D culture.

### ASC Phenotype Characterization

The MSC stem-like phenotype was evaluated for the ASCs at P1/2/6/10 via immunolabel characterization of three key MSC surface markers (CD73/90/105). ASCs were either continuously subcultured in a T-150 flask or allowed to expand within the 3D hydrogel system. At each respective passage time point, cells were seeded in 2D at a standardized density of ˜5000 cells/cm² onto a 96-well glass culture plate (Cellvis, CA, USA; Cat. #P96-1.5H-N) for 2 days, fixed, then assessed for ASC phenotype via immunolabeling for surface CD markers. For ASCs within the 3D culture system, cells were fixed and stained in situ. Positive staining for CD73/90/105 and negative staining for CD34/45 was used to denote a stem-like MSC phenotype for this study. After fixation with 4% paraformaldehyde, cells were washed thrice with HBSS and placed in blocking buffer (1% donkey serum in HBSS) for 1 h. After blocking, primary antibodies for CD73 (Abcam, MA, USA; Cat. #133582; 1:100), CD90 (Abcam; Cat. #181469; 1:100), CD105 (Abcam; Cat. #231774; 1:100), CD34 (Abcam; Cat. #81289; 1:200) or CD45 (Abcam; Cat. #40763; 1:200) were added and the cells incubated overnight at 4° C. The next day cells were washed thrice and secondary antibodies were applied for 1 h, followed by three additional washes. Cells were counterstained with Hoechst 33342 (Invitrogen, MA, USA; Cat. #H3570; 1:1000) and Alexa Fluor R 647 Phalloidin (Invitrogen; Cat. #A22287; 1:1000). Immunofluorescence was assessed with a Revolve microscope using filters for 4′,6-diamidino-2-phenylindole (DAPI; EX-380/30, EM-450/50), fluorescein isothiocyanate (EX-470/40, EM-525/50); Texas Red (EX-560/40, EM-630/75) and Cy5 (EX-630/40, EM-700/75) (Echo, CA, USA) and 20× objective (Olympus, Tokyo, Japan; UPlanSApo, 0.75NA). ASC stem-like phenotypic quantification was carried out in quadruplicate (n=4), with images taken from a total often random fields of view per biological replicate (2D=per well; 3D=per hydrogel), to achieve up to 40 total measured values for each sample. Total nuclei were counted, and total positive cells were evaluated. Phalloidin counterstain was used to aid in localization of positive staining. ASCs characterized at P1 were used as a baseline for comparison.

### ASC Senescence Characterization

Similar to the ASC phenotyping methodology above, assessment of ASC senescence was performed. ASCs were continuously subcultured in a T-150 culture flask until each respective assay time point, when they were seeded onto a 96-well glass culture plate. ASCs in the 96-well plate were allowed to acclimate in serum-based media for 2 days, fixed, then assessed for senescent activity via immunofluorescent labeling of β-galactosidase activity with the CellEvent™ Senescence Green Detection Kit (Invitrogen; Cat. #C10850), per manufacturer's instructions. ASCs in the 3D system were assessed simultaneously at the P2/6/10 passage-equivalent time points. Similar to the method above, cells were counterstained with Hoechst and Phalloidin. Senescence characterization was carried out in quadruplicate (n=4), with images taken from a total of ten random fields of view per biological replicate, to achieve up to 40 total measured values per sample. Total nuclei were counted, and total senescent positive and negative cells were determined.

### Isolation of ASC-Conditioned Media

Media supplementation was standardized for ASC expansion to account for dilutional differences in surface-area-to-volume ratio between 2D and 3D cultures. Media were changed every 2 days. For ASC-conditioned medium (ASC-CM) collection, MSC-GM was removed and cells were washed with HBSS thrice, then cultured with serum-free MSC media for 48 h before collection (for both 2D and 3D cultures). Collected ASC-CM was then centrifuged at 1500×g for 10 min to eliminate cell debris, Steriflip-filtered with a 0.22-μm filter and stored at −80° C. until use.

### KC Activity After ASC-CM Treatment

ASC-CM was collected at each respective time point per the protocol above. ASC-CM was then placed on KCs for up to 24 h to assess its capacity to modulate metabolic, proliferative or migratory activity to evaluate wound healing capabilities of the ASC-CM. For these studies, ASC-CM was added at a 1:1 ratio with KC growth medium. Experimental assays were performed per the manufacturer's instructions. PrestoBlue fluorescence was obtained at 560/590 nm (n=4) and used for evaluation of metabolic activity after 24 h of ASC-CM treatment. Hoechst was added to PrestoBlue samples and values were displayed as average relative fluorescence unit values of PrestoBlue/Hoechst signal in order to control for potential differences in cell numbers and obtain approximate metabolic activity per cell. PicoGreen fluorescence was obtained at 485/535 nm (n=4) and used for evaluation of cell number as a surrogate measurement of KC proliferation after 24 h of ASC-CM culture. Total cell numbers per 96-well plate were calculated based on an average DNA content of 7.7 pg/cell. KC scratch assays were performed to evaluate changes in wound size as a surrogate measurement of KC migration (n=3). Migration images were taken using an ImageXpress Micro XLS Imaging System (Molecular Devices, CA, USA). The entire wound was imaged for each wound triplicate and three different wound regions per wound triplicate were used to calculate wound area. The three wound area values were averaged per triplicate and per time point for each group and displayed as percentage wound area recovered (n=3).

### Statistical Analysis

All data are reported as means with standard error of the mean. Characterization analyses of ASC populations with immunolabeling for senescence and CD markers were evaluated with a two-way analysis of variance. KC metabolic, proliferative and migratory activities were evaluated with a two-way analysis of variance. Data were tested for normality via Shapiro-Wilk and Kolmogorov-Smirnov tests and plotted with a QQ plot. GraphPad Prism 9.0.2 software (GraphPad, CA, USA) was used for the analyses, and a p-value<0.05 was considered significant.

### Results

Unique 3D hydrogel (IPHB) Design Eliminates Subculturing

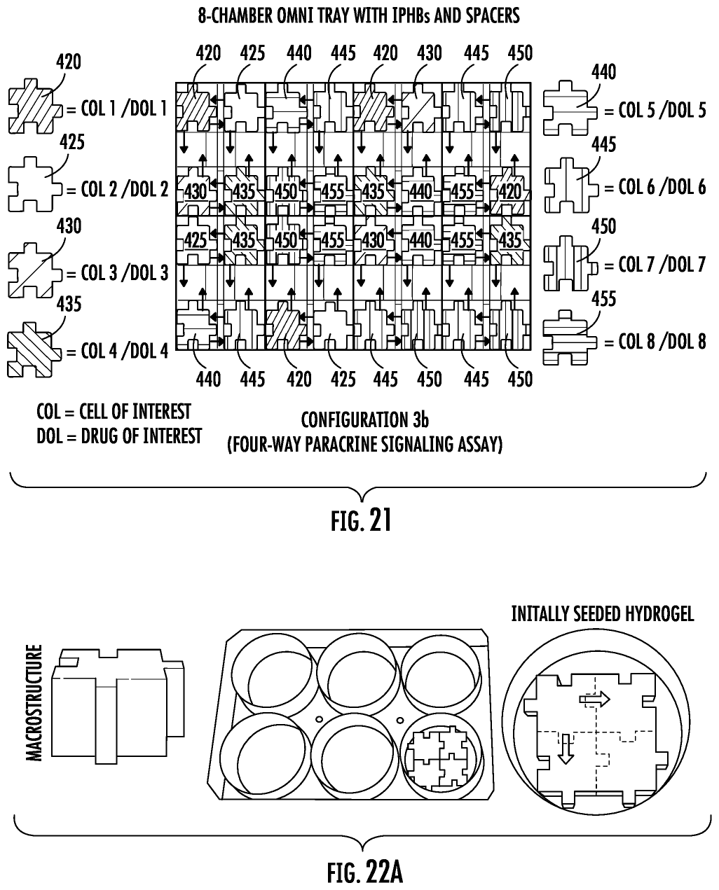

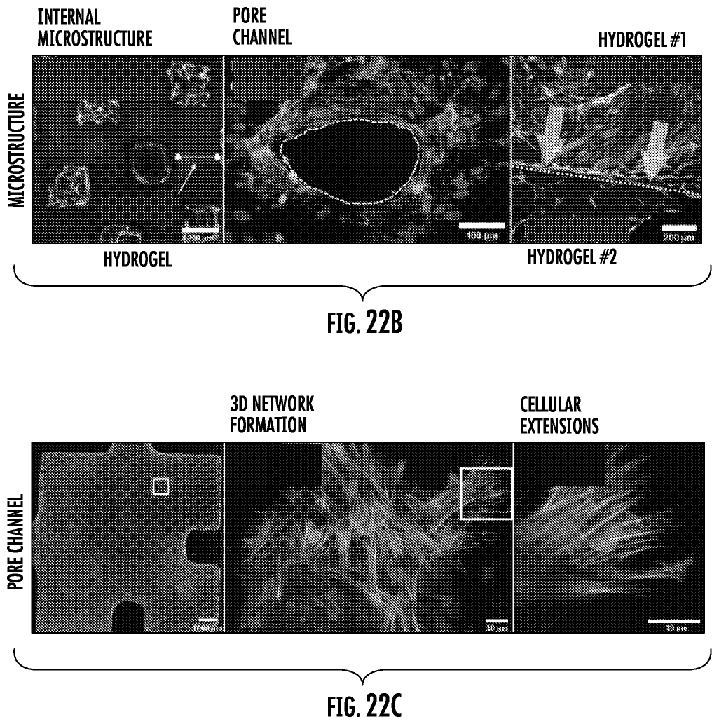

The ˜1-cm³ 3D-printed hydrogel system contains a unique ‘puzzle-piece’ macrostructure that allows the continuous addition of supplementary hydrogels (FIG. 22A) and promotes the migration of cells from the primary seeded hydrogel into the newly attached hydrogels (FIGS. 22A & B). ASCs were cultured within/onto a single hydrogel system for 2 weeks and allowed to migrate throughout the hydrogel. Subsequently, an additional hydrogel was added for 5 days, and cells were allowed to migrate to the newly attached hydrogel. The cells were then stained and assessed for migration and proliferation between the two hydrogels (FIG. 22B). ASCs were seen lining the porous channels beyond the superficial surface within the internal structure and can be seen forming networks within the hydrogel pores (FIG. 22B & C). Cells were able to migrate both across the surface of attached hydrogels and within the microchannels, and can be seen with numerous cellular extensions and focal adhesions protruding from the cells to aid in migration (FIG. 22C). The interaction of ASCs in 3D and the formation of 3D networks within the hydrogel microarchitecture were readily identified. Notably, ASCs could be seen lining the surface of the hydrogel and migrating down into one of the pore channels, ultimately interacting in three dimensions with other cell populations within the pore.

### Retention of Stem-Like MSC Surface Markers in 3D Hydrogel Over Time

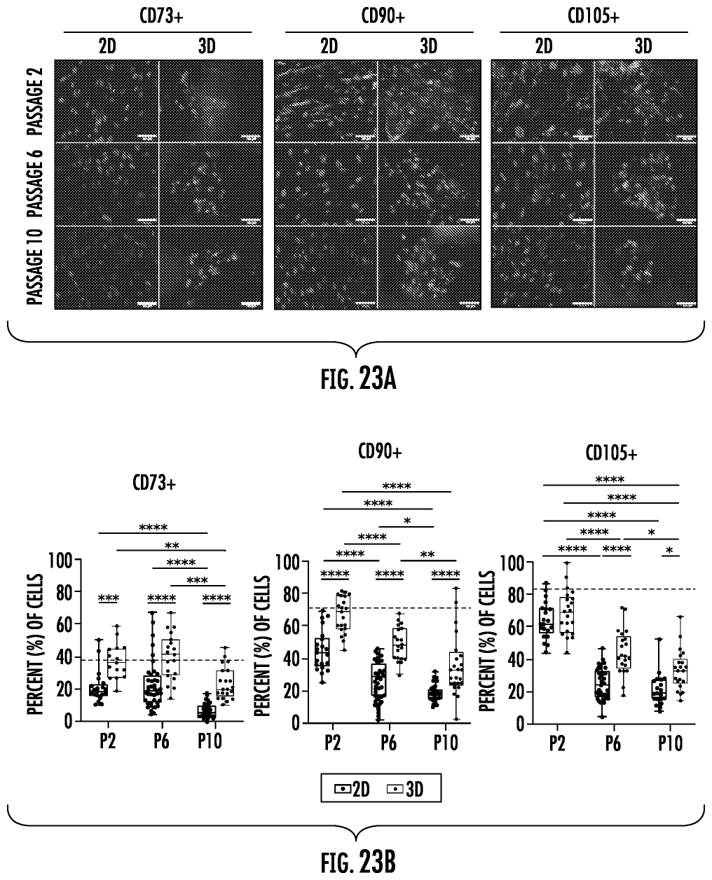

Evaluation of stem-like ASC populations over time was performed via immunolabeling quantification of the CD markers CD73/90/105 (FIG. 23A). The prevalence of stem-like CD markers declined over time in both 2D and 3D systems; however, 2D culture had a significantly enhanced rate of loss of these markers relative to the 3D system (FIG. 23B). Similarly, there was an apparent decrease in intensity of positively stained cells over time in both 2D and 3D systems. Culture within the 3D system resulted in a significant retention at all passage time points for all three positive stem-like markers, except for the initial P2 comparison of CD105. The significant loss of a stem-like phenotype in 2D culture can be seen clearly when comparing ASCs in 3D at P6 and P10 versus ASCs in 2D at P2 (FIG. 23B). Over the course of five or nine passage equivalents in 3D (i.e., 3 or 6 weeks in the 3D system for P6 or P10, respectively) and only one passage event in 2D (seeded at P1), the ASCs had similar expression patterns for multiple CD markers, with only a significant difference between 3D expression of CD105 at P10 relative to 2D CD105 at P2 (FIG. 23B). Both systems maintained a low population (<5%) of positive-staining cells for CD34 and CD45 markers.

### Delayed Induction of Senescence in 3D Hydrogel Over Time

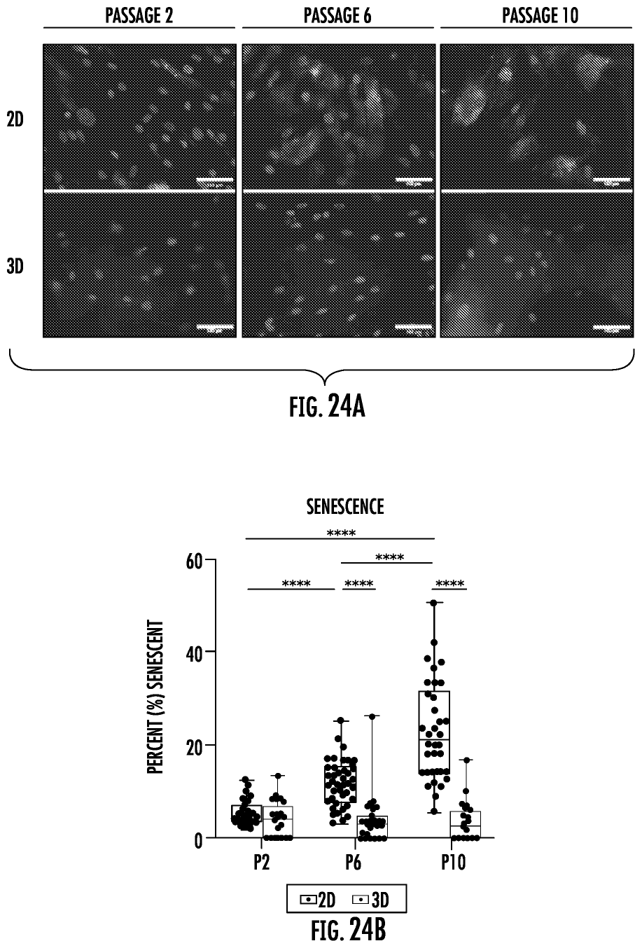

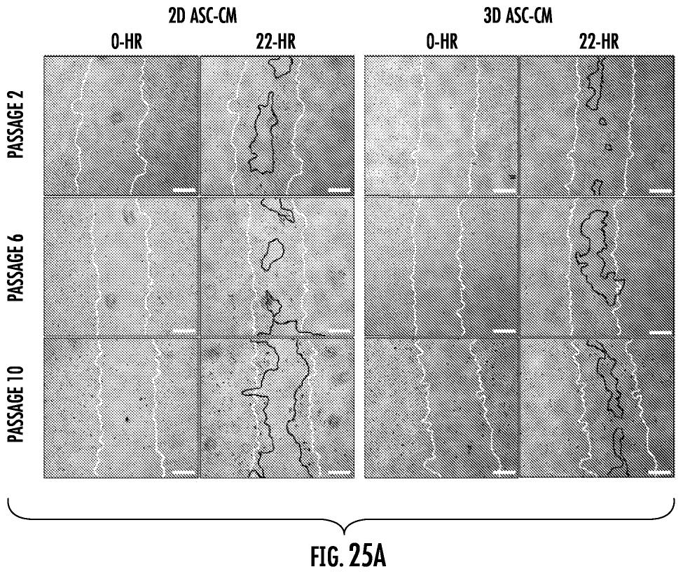

The prevalence of senescent ASC populations over time was evaluated via fluorescent labeling of β-galactosidase activity, a commonly utilized surrogate measurement of cellular senescence (FIG. 24A). The baseline expression of B-galactosidase activity was ˜5% for both 2D and 3D ASCs at P2. Over the course of multiple passaging events in 2D, senescence was significantly increased in 2D, with 11.7 and 22.5% senescent cells at P6 and P10, respectively (FIG. 24B). Conversely, in the 3D system there was no significant change in the prevalence of senescence over time in ASC populations for the time course of this study (6 weeks) (FIG. 24B). Similarly, there were notable differences in cellular morphology in the 2D system versus the 3D system. More specifically, 2D ASCs appear to be more flattened, with a more heterogeneous morphological distribution and an apparent increase in cell size over time in culture, whereas the 3D ASCs maintained a more homogeneous morphology with no observable change in cell size or morphology.

### Retention of ASC Conditioned Media Wound Healing Capacity

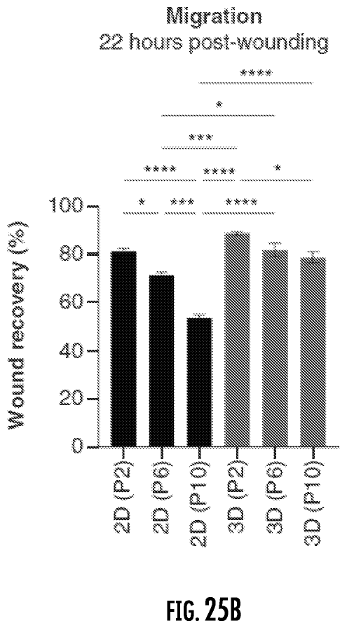

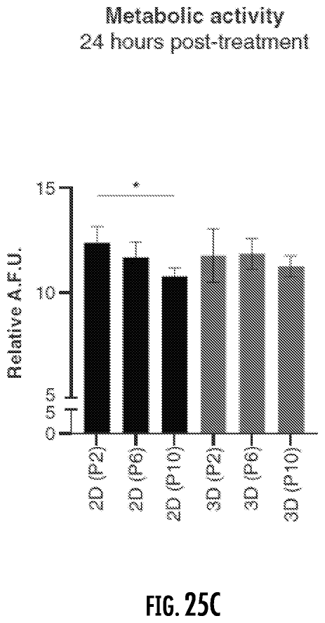

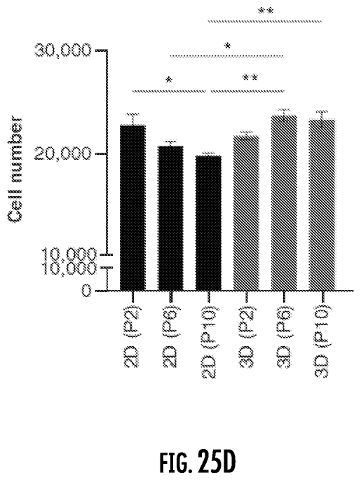

Evaluation of the ASC-CM's functional capacity to modulate a secondary cell population was utilized to demonstrate the dynamic interrelationship between ASC population phenotype and ability to promote wound healing activity in KCs (FIG. 25D). KCs were treated with either 2D or 3D ASC-CM from each respective time point for up to 24 h. KCs treated with ASC-CM from 2D cultures showed a significant drop in recovered wound area when treated with P6 and P10 ASC-CM, relative to P2. There was also a slight decrease noted in KC migratory activity observed with 3D ASC-CM from P10 relative to P2 (FIGS. 25A & B). Overall, 3D ASC-CM maintained a significantly higher ability to enhance KC migration and close their respective wounds, relative to their 2D ASC-CM counterparts. The metabolic activity of KCs demonstrated a decreasing trend when treated with ASC-CM from 2D-expanded cells, with a significant decrease noted in P10 relative to P2 ASC-CM (FIG. 25C). No significant differences were observed with ASC-CM from 3D cultures over time. Similarly, the proliferative activity of KCs treated with ASC-CM from 2D cultures demonstrated a decreasing trend, with a significant difference between P10 and P2 ASC-CM, but no significant change noted when KCs were treated with ASC-CM from 3D cultures (FIG. 25D). Moreover, the proliferative activity of KCs treated with 3D ASC-CM from P6 and P10 was significantly higher than that of their 2D ASC-CM counterparts.

### Discussion

The inherent regenerative properties of MSCs have garnered immense interest for advancing the field of regenerative medicine and tissue engineering. However, MSCs typically must first be removed from a donor tissue source and cultured outside the body within an artificial environment not native to human tissue. To date, commercially available in vitro expansion systems are almost exclusively 2D in nature. Rigid 2D systems are unphysiological for the cells and rapidly result in the loss of MSC multipotent stem-like features, with subsequent loss of viability and induction of senescence. These changes lead to MSC populations with significantly reduced regenerative capabilities, which is compounded by a lack of standardized cell culture conditions, creating a significant bottleneck in the growth and development of regenerative therapeutics. Thus, there is a critical need to develop culture systems for MSC expansion that are 3D and more tissue-mimetic in their mechanical, architectural and substrate composition properties and which can ultimately circumvent many of the limitations of traditional 2D culture, such as the continuous need for subculturing.

Recent advancements in 3D systems have demonstrated progress toward producing MSC populations that are more stem-like. However, 3D systems such as spheroids, organoids, microspheres and many scaffold systems typically do not closely mimic the native mechanics of their cell/tissue source (e.g., adipose mechanics for ASCs) and often require large bioreactor systems and continuous subculturing to achieve large-scale cell numbers for clinical use. However, tissue-engineered hydrogel systems appear to be advantageous toward producing tailorable, tissue-mimetic systems for cell culture systems. More specifically, the mechanotransductive response to the softer substrate of hydrogels is thought to aid in the retention of stem-like characteristics. Unfortunately, most current hydrogel systems are manufactured to promote controlled differentiation of stem cells toward a specific tissue lineage and not to allow the cells to maintain a stem-like phenotype for long-term expansion. Ultimately, an ideal MSC hydrogel expansion system would protect against senescence, while also improving the retention of a regenerative stem-like phenotype and permitting long-term expansion with minimal subculturing or user intervention.

Although MSC-based therapies have demonstrated promise, with over 1000 clinical trials to date listed with the US FDA, they have not advanced as quickly as previously thought. This is considered to be due, at least in part, to the detrimental impact senescent MSC populations may have on tissue regeneration. Senescence is a progressive form of cell-cycle arrest, typically due to DNA and/or oxidative damage, which results in MSCs with impaired DNA-repair modalities that no longer proliferate and exhibit a loss of multipotency. Moreover, senescent MSCs have been shown to secrete factors that negatively impact tissue regeneration and wound healing by impairing angiogenesis, increasing oxidative stress and exacerbating inflammation via the secretion of factors known as the senescence-associated secretory phenotype. The composition of this phenotype can be heterogeneous and is dependent on the mechanism of senescence induction and environmental stimuli; therefore, this likely contributes to the heterogeneity in patient outcomes seen in clinical trials with both cell-based and acellular therapies. Thus, developing an in vitro culture expansion system that limits/prevents the induction of senescence in healthy allogeneic or autologous MSC populations intended for patients would improve the efficacy and consistency of MSC-based clinical therapies.

In this current example, culture of ASCs within a traditional 2D culture system resulted in a significant increase in senescence, as previously established in literature. Conversely, the 3D hydrogel system, in accordance with certain embodiments of the invention, resulted in no significant changes of senescence in ASC populations over the course of the 6-week study (i.e., 10 passage equivalents). Moreover, changes in cell morphology and size can be indicative of phenotypic changes; the apparent increase in cell size seen in the 2D ASCs may be associated with the induction of senescence and thus further supports the B-galactosidase imaging data and prior literature. Overall, these data support the previous hypothesis that 3D culture and substrate mechanics maintain a protective role against senescence. However, with the 3D hydrogel system (IPHB), in accordance with certain embodiments of the invention, the need for subculturing is eliminated, and secreted by-products are more readily accessible versus more traditional poured/molded hydrogels that lack a porous microarchitecture.

Similarly, the stem-like phenotype of MSCs is critical to their regenerative potential and can rapidly change depending on the culture environment of the cells. MSC populations that differentiate and lose their stem-like characteristics result in variability of cellular phenotype and alterations in secretome composition, ultimately decreasing the consistency of regenerative MSC therapeutics, both cellular and acellular. Thus, MSC populations such as ASCs are often used within only a few passaging events in an attempt to circumvent the loss of regenerative potential. However, as we see in this example, even within one additional passaging event in 2D culture, ASCs significantly alter their expression of stem-like markers. Moreover, ASCs expanded in 3D culture for six or ten passaging equivalents (i.e., P6 or P10) over 6 weeks maintained similar expression levels of several markers relative to the baseline P2 ASCs, and a higher expression relative to their respective 2D counterparts, further highlighting the detrimental effects of 2D culture systems on MSC populations and the potential protective effects of 3D culture. The ability to improve the retention of stem-like properties within MSC populations for longer periods of time is desirable for a multitude of applications, including cell therapies, regenerative tissue engineering, immunotherapy and production of secreted biologics.

As previously mentioned, recent MSC therapies have expanded into investigating biologics as a potential regenerative therapy. MSC populations are highly adaptive in nature, and are known to sense their surrounding environmental stimuli and secrete factors accordingly. Thus, secreted bioactive compounds act to coordinate and bridge a variety of tissue reparative processes in an autocrine, paracrine or endocrine manner. In this study, the conditioned media from ASCs cultured in 2D versus the 3D system were collected over time and utilized as a therapeutic for a secondary cell population, KCs, as a means to functionally assess the phenotype of the ASC secretome toward promoting wound healing activity. Working under the hypothesis that the ASC phenotype deteriorates over time in 2D cultures but is sustained in the 3D system, we expected to see a gradual decline in regenerative capabilities of the ASC-CM from 2D cultures but minimal changes in ASC-CM from 3D cultures. This hypothesis was supported by the metabolic, proliferative and migratory data of KCs treated with ASC-CM. ASC-CM from 2D cultures demonstrated a steady decline in ability to augment KC activity and was consistently outperformed by its 3D counterpart over the course of the example.

The limitations of this example include the formulation of the hydrogel system being intentionally bioinert in order to eliminate any contribution of a bioactive substrate, and thus it was not degradable. As a result, adequate removal of cells from this formulation was not feasible. Therefore, immunofluorescent labeling was utilized as an alternative to flow cytometry or RNA analysis to demonstrate senescence and phenotype of MSC populations. However, the ability to perform in situ visualization of an adherent population such as MSCs without the need to resuspend them is an advantage of immunolabeling over cytometry. Moreover, the relative comparison between 2D and 3D cultures, paired with the functional data of the ASC-CM, helps provide a supportive and holistic perspective that reinforces the immunolabeling data, although a more comprehensive analysis of the secretome is needed to assess both qualitative and quantitative changes.

### Conclusion

In this example we illustrate the successful use of a 3D hydrogel system to demonstrate the benefits of culturing MSCs in a system that more closely resembles their native tissue mechanics. The 3D system contains a unique architectural design that does not impede effective mass and fluid transport while also allowing the movement of cells within and between attached hydrogels, in effect providing a continuous 3D culture system that eliminates the need to subculture cells. The continuity is achieved by the addition of supplemental hydrogels to previously seeded hydrogels, much like attaching together two puzzle pieces. The porous microarchitecture creates a ‘tunneling’ system for the cells to interact in the x-, y- and z-planes and to migrate within and between hydrogels, in addition to surface migration at attachment points.

These and other modifications and variations to the invention may be practiced by those of ordinary skill in the art without departing from the spirit and scope of the invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

1-22. (canceled)

23. A method of cultivating cells, comprising:
(i) providing an initial scaffolding comprising a first interlocking porous hydrogel block (IPHB), wherein the first IPHB comprises a three-dimensional (3D) macrostructure defined by a continuous polymeric matrix material and a network of microporous channels and/or chambers extending throughout the continuous polymeric matrix material, and wherein the 3D macrostructure comprises a top surface, a bottom surface, and a thickness defined by at least one side edge extending from the top surface to the bottom surface, and wherein the 3D macrostructure structure includes at least one interlocking-male component and at least one interlocking-female component;
(ii) seeding the first IPHB with one or more cells of interest;
(iii) feeding the one or more cells of interest with a first culture media, and allowing the one or more cells of interest to propagate throughout the network of microporous channels and/or chambers;
(iv) expanding the initial scaffolding by interlocking a second IPHB to the first IPHB, wherein the at least one interlocking-male component or at least one interlocking-female component of the first IPHB is joined to a corresponding interlocking-male component or corresponding interlocking-female component of the second IPHB; and
(v) allowing the one or more cells of interest to propagate from the first IPHB into the second IPHB, and feeding the one or more cells of interest located inside the second IPHB with the first culture media or a second culture media.

24. The method of claim 23, wherein the one or more cells of interest grow and propagate through the network of microporous channels and/or chambers and define a winding pattern wherein the one or more cells of interest wind back-and-forth in a 3D configuration across at least a portion of individual microporous channels as the one or more cells of interest propagate from an interior portion of the first IPHB towards one or more exterior surfaces, such as the top surface, the bottom surface, and/or or the at least one side edge.

25. The method of claim 23, wherein the one or more cells of interest grow and propagate through the network of microporous channels and/or chambers and define a spiral pattern adhered to walls of the network of microporous channels and/or chambers as the one or more cells of interest propagate from an interior portion of the first IPHB towards one or more exterior surfaces, such as the top surface, the bottom surface, and/or or the at least one side edge.

26. The method of claim 23, wherein the one or more cells of interest grow and propagate through the network of microporous channels and/or chambers and define a network of nodes and branches defining open regions therebetween as the one or more cells of interest propagate from an interior portion of the first IPHB towards one or more exterior surfaces, such as the top surface, the bottom surface, and/or or the at least one side edge.

27. The method of claim 23, wherein the one or more cells of interest grow and propagate through the network of microporous channels and/or chambers and define a continuous cell sheet adhered to walls of the network of microporous channels and/or chambers as the one or more cells of interest propagate from an interior portion of the first IPHB towards one or more exterior surfaces, such as the top surface, the bottom surface, and/or or the at least one side edge.

28. The method of claim 23, wherein the one or more cells of interest grow and propagate through the network of microporous channels and/or chambers and define a spheroid.

29. The method of claim 23, wherein the one or more cells of interest grow and propagate through the network of microporous channels and/or chambers and define a comet-like structure having a spheroid structure and a spiral structure emanating from the spheroid structure as the one or more cells of interest propagate from an interior portion of the first IPHB towards one or more exterior surfaces, such as the top surface, the bottom surface, and/or or the at least one side edge.

30. The method of claim 23, further comprising a step of harvesting at least a portion of the one or more cells located throughout the network of microporous channels and/or chambers of the first IPHB.

31. The method of claim 30, wherein the step of harvesting at least a portion of the one or more cells located throughout the network of microporous channels and/or chambers of the first IPHB comprises flushing them out of the first IPHB with a fluid medium or degrading the 3D macrostructure of the first IPHB.

32. The method of claim 23, wherein the method comprises a chain-cultivation method comprising the sequential addition of a plurality of secondary blocks to the first IPHB, wherein the plurality of secondary blocks includes the second IPHB and a third IPHB interconnected directly to the second IPHB such that the second IPHB is located directly between the first IPHB and the third IPHB, and wherein the plurality of secondary IPHBs are initially devoid of cells.

33. The method of claim 32, wherein the cells of interest located in the first IPHB are harvested after cell propagation from the first IPHB to the second IPHB, and the cells of interest located in the second IPHB are harvested after cell propagation from the second IPHB to the third IPHB.

34. The method of claim 23, wherein the method comprises a multi-cell cultivation method, wherein the one or more cells of interest seeded in the first IPHB comprises a first cell type and the second IPHB is seeded with a second cell type, wherein the first cell type is different than the second cell type.

35. The method of claim 34, wherein cells of the first cell type and cells of the second cell type are allowed to propagate towards each other and form a first interface between the first cell type and the second cell type.

36. The method of claim 35 further comprising a step of degrading each of the IPHBs to expose each cell type and the first interface.

37. The method of claim 35, further comprising interlocking a third IPHB directly together with the second IPHB, wherein the second IPHB is located directly between the first IPHB and the third IPHB, and seeding the third IPHB with a third cell type that is different from the first cell type and the second cell type.

38. The method of claim 37, wherein the cells of the second cell type and cells of the third cell type are allowed to propagate towards each other and form a second interface between the second cell type and the third cell type.

39. The method of claim 38, further comprising a step of degrading each of the IPHBs to expose each cell type, the first interface, and the second interface.

40. The method of claim 23, wherein the one or more cells of interest produce or secrete a therapeutic of interest.

41. The method of claim 40, wherein the therapeutic comprises exosomes, extracellular vesicles, growth factors, monoclonal antibodies, peptides, proteins, viral particles, oligonucleotides, organelles, or combinations thereof.

42. The method of claim 23, further comprising a step of coating an interface between the continuous polymeric matrix material and the network of microporous channels and/or chambers extending throughout the continuous polymeric matrix material with a compatibilizer, wherein the compatibilizer is selected to promote adhesion of a primary cell of interest to the IPHB.

43. The method of claim 23, wherein the cultivation method enhances the secretory activity of Mesenchymal Stem Cells (MSCs), improving their regenerative potential for therapeutic applications.
